(19) United States
(12) Patent Application Publication
(10) Pub. No.: US 2018/0298345 A1
(43) Pub. Date: Oct. 18, 2018

(54) ADHESIVE SIGNATURE-BASED METHODS FOR THE ISOLATION OF CANCER-ASSOCIATED CELLS AND CELLS DERIVED THEREFROM

(71) Applicant: Georgia Tech Research Corporation, Atlanta, GA (US)

(72) Inventors: Andres J. Garcia, Atlanta, GA (US); Efrain Cermeno, Atlanta, GA (US); Todd Christopher McDevitt, San Francisco, CA (US); Ankur Singh, Ithaca, NY (US); Susan Napier Thomas, Atlanta, GA (US)

(73) Assignee: Georgia Institute of Technology, Atlanta, GA (US)

(21) Appl. No.: 15/737,973

(22) PCT Filed: Jun. 23, 2016
(86) PCT No.: PCT/US2016/038993

§ 371 Date: Dec. 19, 2017

Related U.S. Application Data
(60) Provisional application No. 62/185,067, filed on Jun. 26, 2015.

(51) Int. Cl. C12N 5/095; C12N 5/071; G01N 33/574

(52) U.S. Cl. CPC C12N 5/0695; C12N 5/0631; G01N 33/574; C12N 2539/10; C12N 2509/10; C12N 2527/00; C12N 2533/52

(57) ABSTRACT

The present invention provides methods of isolating a cancer-associated cell, such as a cancer stem cell or tumor initiating cell or a cell derived therefrom, from a mixture of cells, for example, a mixture of adherent cells in culture. Cell isolation is achieved by the application of selective detachment forces.

### STATEMENT OF PRIORITY

This application claims the benefit of United States Provisional Application Ser. No. 62/185,067, filed Jun. 26, 2015, the disclosure of which is incorporated by reference herein in its entirety.

### STATEMENT OF GOVERNMENT SUPPORT

This invention was made with government support under Grant No. R21 CA202849 awarded by the National Institutes of Health. The government has certain rights in the invention.

### FIELD OF THE INVENTION

The present invention relates to methods for the isolation of cancer-associated cells and cells derived therefrom. In particular, the present invention relates to methods for the isolation of cancer-associated cells and cells derived therefrom based on the use of selective detachment force.

### BACKGROUND OF THE INVENTION

Tumors are heterogeneous tissues that contain a small population of stem-like cells that self renew, differentiate into various cancerous progeny types, and survive hostile microenvironments to form tumors. Such tumor-initiating cells (TICs) have been identified in cell lines and patient samples using surface markers and their ability to generate tumor spheres and xenograft tumors. However, TIC sub-populations from various sources differ greatly in their surface marker expression profile, and, to date, there is no universal marker profile to identify TICs. This inability to effectively isolate TIC sub-populations with high purity/yield is a profound impediment to characterizing the biology of these cells as well as analyzing patient biopsies for effective diagnosis or prognosis.

The present invention overcomes previous shortcomings in the art by providing adhesive-signature based methods for isolation of specific cancer-associated cell populations.

### SUMMARY OF THE INVENTION

The present invention is based, in part, on the inventors' demonstration of a unique “adhesive signature” associated with cancer-associated cells (e.g., tumor initiating cells, cancer stem cells, cancer stem-like cells) and cells derived therefrom, which is dictated by their phenotypic state. The present invention utilizes the differences in the adhesion strength of such cancer-associated cells, as well as cancer-associated cell derivatives, as compared with other cells (e.g., other cancer-associated cells or other cancer cells or other non-cancer cells) to selectively isolate cell type(s) of interest using detachment forces. Advantageously, the methods of the invention are amenable to high throughput analysis, real-time imaging, in-line biochemical, genetic and/or cytometric processing.

Thus, in one aspect, the present invention provides a method of isolating a cancer-associated cell (e.g., a cancer stem cell, a tumor initiating cell or a cancer stem-like cell) from a mixture of cultured animal cells, comprising subjecting a mixture of cultured animal cells adhered to a substrate comprising the cancer-associated cell and at least one other cell type to a detachment force that is sufficient to selectively detach the cancer-associated cell from the substrate relative to the at least one other cell type in the mixture of cultured animal cells, thereby isolating the cancer-associated cell from the mixture of cultured animal cells.

The foregoing and other aspects of the present invention will now be described in more detail with respect to other embodiments described herein.

### BRIEF DESCRIPTION OF THE DRAWINGS

FIG. 1. Adhesion signature strength of human induced pluripotent stem cells (hiPSCs) undergoing reprogramming and differentiation. (Panels A and B) Adhesion strength of cells during reprogramming (Panel A) and the indicated cell types on fibronectin (FN) and laminin (LM) (Panel B). (Panel C) Adhesion strength for undifferentiated (UD) and spontaneously differentiating (SD) cultures of hiPSCs and human embryonic stem cells (hESCs) on FN or LM.

FIG. 2. Adhesion strength-based isolation of pluripotent stem cells in microfluidic devices. (Panels A and B) Enrichment of hiPSCs and hESCs isolated at 85-125 dynes cm ⁻² from a coculture with IMR90 and mouse embryonic fibroblast cells, respectively. Graphs show mean±s.d. (*P<0.05, n=3).

FIG. 3. Continued culture and expansion of hiPSCs in μSHEAR platform. (Panels A and B) The hiPSCs can be expanded within the microfluidic devices while maintaining equal or higher degrees of purity (Panel A) and survival (Panel B) than conventional methods of purification.

FIG. 4. Cell counts and mammosphere characterization following 10 day MFA after adhesion force separation. (Panel A) Cells that strongly attach to the matrix produce larger mammospheres after a 10 day MFA. (Panel B) Mammospheres were disassociated into single cells and counted. The fraction of cells that attached the strongest (RC) displayed a 5-15 fold increase in the number of cells at the 10 day time point compared to the 0.8-1.7 fold increase seen in the controls. Furthermore, as the selection adhesive force for the RC fraction was increased, greater increases in the number of cells were seen. (Panel C) Mammosphere counts with 185.3 dynes cm⁻² of shear force used for separation. Cells that adhere more strongly to the matrix produce mammospheres with both a larger number of proliferative cells and a larger size.

FIG. 5. Adhesion strength of different cell populations. (Panel A) Representative spinning disk detachment profiles. Cells were grown on fibronectin-coated coverslips. After 24 hr, spinning disk experiments were performed and the adhesion strength was measured. (Panel B) A significant difference in adhesion is seen between immortalized hTERT-HME1 and the MDA cancer lines. (Panel C) Nonlinear fit of MDA-MB-453 detachment values after shear force application in microfluidic devices. The shear force values used in the remaining experiments are highlighted.

FIG. 6. Cell and mammosphere counts following 10 day culture in MFA after adhesion force separation. (Panel A) Mammospheres were dissociated into single cells and counted. The fraction of cells that attached the strongest (RC) displayed a 5-15 fold increase in the number of cells at the 10 day time point compared to the 0.8-1.7 fold increase seen in the controls. Furthermore, as the selection adhesive force for the RC fraction was increased, greater increases in the number of cells were seen. (Panels B-D) Mammosphere counts with varying degrees of shear force used for purification: 58.1 dynes/cm² (Panel B), 105.3 dynes/cm² (Panel C), and 185.3 dynes/cm² (Panel D). Cells that adhere more strongly to the matrix produce mammosphere both a larger number of proliferative cells and larger size.

FIG. 7. Quantification of mammosphere size. (Panels A-C) Histograms of the mammospheres radii for cells separated with 58.1 dynes/cm² (Panel A), 105.3 dynes/cm² (Panel B), and 185.3 dynes/cm² (Panel C). The probability distribution of the RC fraction in panel C is significantly different than the others.

FIG. 8. Schematic of μSHEAR protocol.

FIG. 9. The adhesive strength signature of breast cancer cells varies significantly not only among cell lines, but also within them. (Panel A) Spinning disk adhesive force measurements of a panel of breast cancer cell lines and the hTERT-HME1 immortalized mammary cell line. Statistical analysis was performed using one way ANOVA. (Panel B) μSHEAR detachment profiles of MDA-MB-231 and MDA-MD-453 cells.

FIG. 10. Enrichment of MDA-MB-453 cells with increased mammosphere formation capabilities. MDA-MB-453 cells were introduced into the μSHEAR devices and exposed to three levels of shear forces. Detached cells as well as those that remained attached were collected and seeded into an MFA. Two samples were performed for the 58.1 dynes/cm² target shear stress. The radius of the resulting mammospheres was quantified.

FIG. 11. B16 melanoma xenograft tumor generation and isolation. (Panel A) eGFP-B16 melanoma cells were generated by lentiviral infection. Xenograft tumors were generated in NOD/SCID mice using eGFP B16 melanoma cells. After 10 days, cells were isolated and introduced into the μSHEAR device. Both B16 cancerous eGFP+cells (38.5%) as well as non-cancerous eGFP-cells (61.5%) survived the procedure.

FIG. 12. (Panel A) Flow cytometry plots showing detached hiPSC (TRA-1-60+/CMPTX+) and IMR90 cells (TRA-1-60−/CMPTX+). At 85-125 dynes/cm² shear stress, hiPSC were isolated with 99% purity, while at 250 dynes/cm² both hiPSC and IMR90 cells detached. (Panel B) Enrichment efficiency of hiPSC when repeatedly passaged by μSHEAR, EDTA, TrypLE, Dispase, or Accutase over the course of 10 passages (*p<0.05).

FIG. 13. μSHEAR-based isolation of hiPSC from a heterogeneous reprogramming culture. (Panel A) Left, analysis of an unpurified reprogramming culture in devices with baseline 0.65% hiPSC purity. Center, flow cytometry plot showing detached hiPSC (TRA-1-60+CMPTX+) and nonreprogrammed/partially reprogrammed cells (TRA-1-60−CMPTX+). Right, analysis of residual cells in the device after μSHEAR. (Panel B) Hematoxylin & eosin (H&E) stained sections from a teratoma produced from μSHEAR-isolated hiPSCs showing cartilage (mesoderm) and glands (endoderm).

FIG. 14. Schematic of μSHEAR microfluidics device and scale-up.

### DETAILED DESCRIPTION OF THE INVENTION

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a method comprises steps A, B and C, it is specifically intended that any of A, B or C, or a combination thereof, can be omitted and disclaimed singularly or in any combination. As another example, if the specification states that a cell has particular characteristics, X, Y and Z, it is specifically intended that any of X, Y, Z, or a combination thereof, can be omitted and disclaimed singularly or in any combination.

As used herein, “a,” “an” or “the” can mean one or more than one. For example, “a” cell can mean a single cell or a multiplicity of cells.

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” when used in a claim herein is not intended to be interpreted to be equivalent to “comprising.”

The present invention is based on the unexpected discovery that certain sub-populations of cancer cells can be isolated using detachment forces. Thus, in one embodiment, the present invention provides a method of isolating a cancer-associated cell from a mixture of cultured animal cells, comprising subjecting a mixture of cultured animal cells adhered to a substrate, the mixture comprising the cancer-associated cell and at least one other cell type, to a detachment force that is sufficient to selectively detach the cancer-associated cell from the substrate relative to the at least one other cell type in the mixture of cultured animal cells, thereby isolating the cancer-associated cell from the mixture of cultured animal cells. In particular, in the present invention, it has been shown that adhesion-based cancer-associated cell (e.g., TIC) enrichment outperforms surface-marker based enrichment.

As used herein, the term “cancer-associated cell” refers to a cancer stem cell (CSC), a tumor initiating cell (TIC) or a cancer stem-like cell (CSLC). A common feature of any of these cancer-associated cells is the ability to initiate new tumors in immunocompromised mice. In some embodiments, the cancer-associated cells of this invention have increased drug resistance and/or mammosphere formation capability relative to a cancer cell that lacks the ability to initiate new tumors in immunocompromised mice or to a non-cancer cell (e.g., a normal cell).

In the methods described herein, the “at least one other cell type” refers to a cell that is not a cancer-associated cell (i.e., not a CSC, TIC or CSLC) of this invention, but can be a stem cell, a progenitor cell, a terminally differentiated cell, a stromal cell, an inflammatory cell, an explant cell, a non-TIC cancer cell and/or a progeny cell of any of these cells. In particular embodiments, at least one other cell type will have adhesion properties that are sufficiently different from the adhesion properties of the cancer-associated cell to allow for isolation of the cancer-associated cell from a mixture of cells comprising both the cancer-associated cell and the at least one other cell type. In some embodiment, it may be desirable to isolate a cell of interest that is not a cancer-associated cell from a mixture of cells comprising a cancer-associated cell and the cell of interest and therefore, the present invention provides such a method of isolating a cell that is not a cancer-associated cell from a mixture of cultured animal cells that comprises cancer cells, comprising subjecting a mixture of cultured animal cells adhered to a substrate, the mixture comprising the non-cancer-associated cell and at least one other cell type that is a cancer-associated cell or non-TIC cancer cell, to a detachment force that is sufficient to selectively detach the cancer-associated cell from the substrate relative to the at least one other cell type in the mixture of cultured animal cells, thereby isolating the cancer-associated cell not associated with cancer (e.g., a non-cancer cell) from the mixture of cultured animal cells.

In some embodiments of the methods of this invention, the cancer-associated cell can grow in culture as part of a cluster and in some embodiments, the cancer-associated cell can detach from the substrate as part of a cluster of cancer-associated cells.

In the embodiments of this invention, the detachment force that is sufficient to selectively detach the cancer-associated cell provides a wall shear stress in the range of about 20 to about 1500 dynes/cm².

In some embodiments, the cancer-associated cell detaches at a lower detachment force as compared with the at least one other cell type, and in some embodiments, the cancer-associated cell detaches at a higher detachment force as compared with the at least one other cell type.

In some embodiments of this invention, the isolated cancer-associated cell is viable and in some embodiments, the isolated cancer-associated cell can maintain the ability to divide and produce progeny cells and/or form tumors.

In some embodiments of this invention, a plurality of cancer-associated cells can be isolated with at least about 70% (e.g., 50%, 60%, 70%, 80%, 90%, 95%, 100%) purity.

In particular embodiments of this invention, at least 70% (e.g., 50%, 60%, 70%, 80%, 90%, 95%, 100%) of the cancer-associated cells in the mixture of cultured animal cells are isolated.

Cells used in carrying out the present invention (e.g., the “cultured animal cells”) are, in general, animal cells including mammalian cells and/or avian cells. Mammalian cells include but are not limited to human, non-human mammal, non-human primate (e.g., monkey, chimpanzee, baboon), dog, cat, mouse, hamster, rat, horse, cow, pig, rabbit, sheep and goat cells. Avian cells include but are not limited to chicken, turkey, duck, geese, quail, and pheasant cells, and cells from birds kept as pets (e.g., parakeets, parrots, macaws, cockatoos, and the like). In particular embodiments, the cell is from a species of laboratory animal Suitable animal cells include cells from both males and females and animals of all ages including embryonic, infant, neonatal, juvenile, adolescent, adult and geriatric animals.

A “mixture of animal cells” or “mixture of cultured animal cells” refers to two or more types of animal cells (e.g., 2, 3, 4, 5, 6 or more). According to embodiments of the present invention, the mixture of animal cells is a mixture of adherent animal cells (e.g., in culture).

The “cell of interest” or “cell type of interest” as used herein refers to a cell or cell type that it is desired to be isolated according to the methods of this invention, but is not indicative of the intended use of the cells. For example, in embodiments, the “cell of interest” to be isolated can be a contaminating cell (e.g., a non-cancer cell or non-TIC cell in a culture of cells comprising non-cancer cells and/or non-TIC cells as well as cancer-associated cells), which optionally may be discarded.

“Adhesion strength” as used herein refers to the strength with which a cell is attached (e.g., adhered) to a substrate and is proportional to the shear stress required to separate the cell therefrom. Adhesion strength of a cell to the substrate is a function of a number of properties including the quantity and spatial distribution of adhesion receptors and the association of bound integrins to cytoskeletal elements. In embodiments, if one cell has a “higher,” “greater” or “increased” (and like terms) adhesion strength as compared with another cell, the adhesion strength is at least about 1.2, 1.5, 2, 3, 4, 5, 6, 7, 8, 9 or 10-fold higher (e.g., as determined by detachment force). In embodiments, if one cell has a “lower,” “lesser” or “reduced” (and like terms) adhesion strength as compared with another cell, the adhesion strength of the first cell is less than about 70%, 60%, 50%, 40%, 30%, 20%, 10% or less than that of the second cell.

The term “substrate” as used herein refers to the surface on which the cells are adhered (e.g., cultured). The substrate can be glass and/or plastic. Examples of suitable substrates include without limitation slides, cover slips, culture dishes, culture bottles, multi-well plates and/or a cassette that fits into a device (e.g., for use with a microfluidic device). The “substrate” can optionally be coated, e.g., with an extracellular matrix protein, including without limitation, laminin, collagen (e.g., collagen IV), vitronectin, fibronectin, entactin, and/or a synthetic polymer coating such as poly[2-methacryloyloxy)ethyl dimethyl-(3-sulfopropyl) ammonium hydroxide] (PMEDSAH), and/or other biological molecules such as antibodies, aptamers, and cell-cell receptor proteins (e.g., cadherins). Suitable extracellular matrix formulations are commercially available, such as isvitronectin (R&D Systems), MATRIGEL™ and Laminin-511. As a further option, feeder cells can be grown on the substrate. In some embodiments, a microgrooved or chemically patterned surface can be included in the μSHEAR device of this invention.

The term “detachment force” as used herein refers to a force that is sufficient to detach, remove or separate a cell from the substrate on which it is adhered. The detachment force can be applied by any suitable method including, without limitation, hydrodynamic force, centrifugal force and/or magnetic force. The detachment force can optionally be described in terms of the force that produces a shear stress (τ, force/area) that results in 50% detachment of a plurality of the cells (τ₅₀). In embodiments, the detachment force provides a wall shear stress that is greater than about 10, 20, 30, 40 or 50 dynes/cm² and/or less than about 100, 105, 110, 115, 120, 125, 130, 140, 150, 160, 170, 180, 190, 200, 225, 250, 300, 350, 400 or 500 dynes/cm² (including all combinations of lower and higher values as long as the lower limit is less than the upper limit). In embodiments, the detachment force provides a wall shear stress that is from about 20 to about 40, 50, 60, 70, 80, 90, 100, 105, 110, 115, 120, 125, 130, 140, 150, 160, 170, 180, 190, 200, 225, 250, 300, 350 or 400 dynes/cm². In embodiments, the detachment force provides a wall shear stress that is from about 30 to about 40, 50, 60, 70, 80, 90, 100, 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 40 to about 50, 60, 70, 80, 90, 100, 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 50 to about 60, 70, 80, 90, 100, 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 60 to about 70, 80, 90, 100, 110, 105, 110, 115, 120, 125, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 70 to about 80, 90, 100, 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 80 to about 90, 100, 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 90 to about 100, 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 100 to about 105, 110, 115, 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 110 to about 120, 125, 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 120 to about 130, 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 130 to about 140, 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 140 to about 150, 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 150 to about 160, 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 160 to about 170, 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 170 to about 180, 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 180 to about 190, 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 190 to about 200, 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 200 to about 225, 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 225 to about 250, 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 250 to about 300, 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 300 to about 350, 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 350 to about 400, 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 400 to about 450, 500, 550, 600, 650, 700, 750, 800, 850, 900, 950, 1000, 1100, 1200, 1300, 1400 or 1500 dynes/cm². In embodiments, the detachment force provides a wall shear stress that is from about 450 to about 500, 550, 600, 650, 700, 750, 800, 850, 900, 950, 1000, 1100, 1200, 1300, 1400 or 1500 dynes/cm². In embodiments, the detachment force provides a wall shear stress that is from about 500 to about 550, 600, 650, 700, 750, 800, 850, 900, 950, 1000, 1100, 1200, 1300, 1400 or 1500 dynes/cm². In embodiments, the detachment force provides a wall shear stress that is from about 550 to about 600, 650, 700, 750, 800, 850, 900, 950, 1000, 1100, 1200, 1300, 1400 or 1500 dynes/cm². In embodiments, the detachment force provides a wall shear stress that is from about 600 to about 650, 700, 750, 800, 850, 900, 950, 1000, 1100, 1200, 1300, 1400 or 1500 dynes/cm². In embodiments, the detachment force provides a wall shear stress that is from about 650 to about 700, 750, 800, 850, 900, 950, 1000, 1100, 1200, 1300, 1400 or 1500 dynes/cm². In embodiments, the detachment force provides a wall shear stress that is from about 700 to about 750, 800, 850, 900, 950, 1000, 1100, 1200, 1300, 1400 or 1500 dynes/cm². In embodiments, the detachment force provides a wall shear stress that is from about 750 to about 800, 850, 900, 950, 1000, 1100, 1200, 1300, 1400 or 1500 dynes/cm². In embodiments, the detachment force provides a wall shear stress that is from about 800 to about 850, 900, 950, 1000, 1100, 1200, 1300, 1400 or 1500 dynes/cm². In embodiments, the detachment force provides a wall shear stress that is from about 850 to about 900, 950, 1000, 1100, 1200, 1300, 1400 or 1500 dynes/cm². In embodiments, the detachment force provides a wall shear stress that is from about 900 to about 950, 1000, 1100, 1200, 1300, 1400 or 1500 dynes/cm². In embodiments, the detachment force provides a wall shear stress that is from about 950 to about 1000, 1100, 1200, 1300, 1400 or 1500 dynes/cm². In embodiments, the detachment force provides a wall shear stress that is from about 1000 to about 1100, 1200, 1300, 1400 or 1500 dynes/cm². In embodiments, the detachment force provides a wall shear stress that is from about 1100 to about 1200, 1300, 1400 or 1500 dynes/cm². In embodiments, the detachment force provides a wall shear stress that is from about 1200 to about 1300, 1400 or 1500 dynes/cm². In embodiments, the detachment force provides a wall shear stress that is from about 1300 to about 1400 or 1500 dynes/cm². In embodiments, the detachment force provides a wall shear stress that is from about 1300 to about 1400 or 1500 dynes/cm². In embodiments, the detachment force provides a wall shear stress that is from about 1400 to about 1500 dynes/cm². Further, the detachment force can be applied as a consistent force or can be variable (e.g., within a range).

As used herein, “selectively detach” (and similar terms) refers to preferential detachment of a particular cell type within a mixture of cells from a substrate to which the cell is adhered as compared with at least one other cell type in the mixture of cells adhered to the substrate. In embodiments of the invention, to achieve selective detachment the wall shear stress that results in 50% detachment (τ₅₀) of a cell type of interest (e.g., a cancer-associated cell) is at least about 1.2, 1.5, 2, 3, 4, 5, 6, 7, 8, 9 or 10-fold lower or higher as compared with the 1₅₀ for at least one other cell type in a mixture of adherent cells. Thus, the cell of interest to be isolated can selectively detach with a higher or lower τ₅₀ than the at least one other cell type in the mixture of cells. In embodiments, at least about 50%, 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold or more of the cell type of interest (e.g., a cancer-associated cell) detaches relative to the at least one other cell type. In representative embodiments, the detachment force that “selectively detaches” a particular cell type as compared with at least one other cell type in a mixture of cells adhered to a substrate results in at least about 60%, 70%, 75%, 80%, 85%, 90%, 95%, 96%, 97%, 98%, 99% or more detachment of the first cell type and/or less than about 40%, 30%, 25%, 20%,15%, 10%, 5%, 4%, 3%, 2%, 1% or less detachment of at least one other cell type in the mixture of cells from the substrate.

As used herein, an “isolated” cell produced by a method of the invention is a cell that has been partially or completely separated, enriched and/or purified from other components (e.g., cells of other types in the mixture of cells) with which it is associated in the mixture of cells (e.g., adherent cells in culture) prior to the use of the methods of the invention. Those skilled in the art will appreciate that an “isolated” plurality or population of cells need not be 100% pure, as long as there is some enrichment or increase in the concentration of the cells of interest as compared with the concentration of the cells in the starting material prior to the use of the methods of the invention. In embodiments, the concentration of the “isolated” cell is increased by at least about 2-fold, 3-fold, 4-fold, 5-fold, 10-fold, 20-fold, 30-fold, 40-fold, 50-fold, 60-fold, 80-fold, 100-fold, 150-fold, 200-fold, 300-fold, 400-fold, 500-fold, 600-fold, 800-fold, 1000-fold or more by the practice of the methods of the invention. In embodiments of the invention, an “isolated” plurality or population of cells is at least about 50%, 60%, 70%, 75%, 80%, 85%, 90%, 95%, 96%, 97%, 98%, 99% or more pure.

“Totipotent” as used herein, refers to a cell that has the capacity to form an entire organism.

“Pluripotent” as used herein refers to a cell that has essentially complete differentiation versatility, e.g., the capacity to grow into essentially any of the animal's cell types (e.g., cells derived from any of the three germs layers: endoderm, mesoderm and ectoderm). A pluripotent cell can be self-renewing, and can remain dormant or quiescent. Unlike a totipotent cell, a pluripotent cell cannot usually form a new blastocyst or blastoderm. A pluripotent cell generally expresses one or more pluripotency markers. Markers of pluripotency are well known in the art and include, without limitation: OCT4 (POU5F1), NANOG, SOX2, SSEA4 (human), SSEA1 (mouse), SSEA3, TRA-1-60, TRA-1-81, alkaline phosphatase, CD30 (Cluster Designation 30), GCTM-2, Genesis, germ cell nuclear factor, telomerase, and Rex-1 (these terms also encompass homologs from other species).

“Multipotent” as used herein refers to a cell that has the capacity to produce any of a subset of cell types of the corresponding animal (e.g., two or more cell types). Unlike a pluripotent cell, a multipotent cell does not have the capacity to form all of the cell types of the corresponding animal Examples of multipotent cells include lineage committed cells and progenitor cells. Markers associated with particular lineages are well-known in the art and include, without limitation: neural markers (e.g., Nestin, CD133, and/or Musashi-1), hematopoietic markers (e.g., CD34 and/or c-Kit), pancreatic lineage marker (e.g., Nestin and/or vimentin), skeletal muscle markers (e.g., MyoD, Pax7, myogenin, MR4 and/or myosin light chain), cardiac muscle markers (e.g., MyoD, Pax7, and/or myosin heavy chain), and the like.

As used herein, the term “stem cell” includes without limitation: embryonic stem (ES) cells (e.g., derived from the epiblast tissue of the inner cell mass of a blastocyst or earlier morula stage embryo and/or produced by somatic cell nuclear transfer), an induced pluripotent stem (iPS) cell and/or an adult stem cell (e.g., a somatic stem cell and/or a germ line stem cell). In embodiments of the invention, the stem cell is not an adult stem cell. Stem cells are generally characterized by the capacity for self-renewal (the ability to undergo numerous cycles of cell division while maintaining an undifferentiated state) and pluripotency or, in some cases, multipotency. In embodiments of the invention, the stem cell grows in clusters of at least about 2, 4, 6, 8, 10, 20, 40, 60, 80, 100 or more cells (e.g., cells connected by cell-cell adhesions or junctions). In embodiments, the stem cell exhibits apoptosis when not grown or cultured in a cell cluster.

An “undifferentiated stem cell” is generally a pluripotent or multipotent cell. Those skilled in the art will appreciate that ES cells and iPS cells are typically considered pluripotent and express one or more (e.g., 1, 2, 3, 4, 5 or more) pluripotency markers (as that term is understood in the art and as described herein). On the other hand, adult stem cells are typically multipotent, and express one or more markers (e.g., 1, 2, 3, 4, 5 or more) associated with particular lineages. However, some adult stem cells are pluripotent (e.g., stem cells isolated from umbilical cord blood), and can express one or more markers (e.g., 1, 2, 3, 4, 5 or more) associated with pluripotency. Adult stem cells are often referred to by their tissue of origin; mesenchymal stem cells, hematopoietic stem cells, adipocyte-derived stem cells, endothelial stem cells and dental pulp stem cells are nonlimiting examples of adult stem cells.

A cell “derived from a stem cell” and similar terms as used herein refers to cells that are produced from stem cells (e.g., undifferentiated stem cells) as a result of differentiation processes. Such cells include without limitation, spontaneously differentiated and directly differentiated stem cells (e.g., lineage committed cells, progenitor cells and/or terminally differentiated cells) and cells in intermediate stages of differentiation. Those skilled in the art will appreciate that the process of differentiation into different cell types from a stem cell is a continuum and cells with intermediate characteristics are often present.

A “spontaneously differentiated stem cell” or “spontaneously differentiated cell” as used herein is a cell derived from an undifferentiated stem cell as a result of a spontaneous (e.g., not directed) differentiation process. Spontaneously differentiated cells are a problematic contaminant of stem cell cultures and pose an obstacle to the culture and use of cultured stem cells. “Spontaneously differentiated stem cells” or “spontaneously differentiated cells” appear to differentiate along random pathways and generally have reduced pluripotency and reduced expression of at least one pluripotency marker as compared with undifferentiated stem cells. In some instances, “spontaneously differentiated stem cells” appear as spread, fibroblast-like cells.

The term “directly differentiated stem cell” or “directly differentiated cell” refers to a cell that has been directed to differentiate along a particular pathway, e.g., by manipulation of culture medium components. Directly differentiated cells include lineage committed cells, progenitor cells, and terminally differentiated cells as well as cells in intermediate stages of differentiation.

The term “lineage committed cell” as used herein indicates a cell that has begun to express markers and/or exhibit morphology, structure, potency (e.g., the ability to differentiate along a particular lineage(s)) and/or other characteristics associated with a particular lineage, but is not yet a “progenitor” cell. Thus, “lineage committed cells” can be viewed as intermediates between stem cells and progenitor cells. Examples of lineage—committed cell include without limitation a neural committed cell (e.g., a neural rosette cell), a hematopoietic committed cell, a skeletal muscle committed cell, a cardiac muscle committed cell, a pancreatic committed cell, and the like. As one illustration, neural rosette cells express the protein marker nestin, but grow as radial clusters, whereas neural progenitor cells grow as individual elongated cells. Thus, neural rosette cells express intermediate characteristics between stem cells and neural progenitor cells.

A “progenitor cell” as used herein refers to a multipotent cell that typically can divide only a limited number of times prior to terminal differentiation. “Progenitor cells” are early descendents of stem cells that typically have a reduced potency and self-replication capacity as compared with stem cells. Nonlimiting examples of progenitor cells include neural progenitor cells, hematopoietic progenitor cells, cardiac muscle progenitor cells, skeletal muscle progenitor cells, pancreatic progenitor cells, and the like.

The term “feeder” cell is well-known in the art and encompasses cells (e.g., fibroblasts, bone marrow stromal cells, and the like) that are cultured with other cells (for example, stem cells) and support the viability and/or growth thereof.

The term “parental somatic” cell or “parental” cell refers to a cell that is reprogrammed to produce an iPS cell. As is known in the art, iPS cells are derived from other, typically non-pluripotent, cells such as a somatic cell (e.g., an adult somatic cell such as a fibroblast) by inducing expression of particular genes and/or introducing particular nucleic acids and/or proteins that result in reprogramming of the cell. iPS cultures are frequently contaminated by non-pluripotent parental cells and/or partially reprogrammed cells. The parental cells can generally be identified by methods known in the art, e.g., morphology (elongated) and/or reduced expression or lack of expression of one or more pluripotency markers (as known in the art and as described herein). Typically, partially reprogrammed cells have taken up some, but not all, of the reprogramming factors (e.g., are transformed with some but not all of the nucleic acids introduced to reprogram the cells). In addition, partially-reprogrammed cells often have a rounded or less-spread morphology as compared with the parental cells, but generally do not express pluripotency markers.

The inventors have made the surprising discovery that the characteristic “adhesive signature” associated with cancer-associated cells (e.g., CSC, TICs, CSLCs) and derivatives thereof can be used to selectively detach and isolate these cells from each other and/or from other cells in a mixture of animal cells adhered to a substrate based on differences in adhesion strength for the substrate on which the cells are adhered (e.g., cultured). A cancer-associated cell can be isolated from a mixture of cells adhered to a substrate if there is a sufficient difference (higher or lower) in the adhesion strength of the cancer-associated cell to the substrate relative to at least one other cell type (e.g., a non-cancerous cell or non-TIC cell) present in the mixture of cells, such that a detachment force can be applied that will selectively detach the cancer-associated cell from the substrate as compared with the at least one other cell type in the mixture of cells adhered to the substrate.

In embodiments, the cancer-associated cell selectively detaches at a lower detachment force from the substrate as compared with at least one other cell type (e.g., 1, 2, 3, 4, 5 or more other cells types) in the mixture of cells. Nonlimiting examples include the selective detachment of cancer-associated cells from a mixture of cells that comprises cancer-associated cells, non-cancer cells and/or non-TIC cells.

In embodiments, the cell of interest (e.g., a cancer-associated cell) selectively detaches from the substrate at a higher detachment force as compared with at least one other cell type (e.g., 1, 2, 3, 4, 5, 6, 7, 8, 9, 10, etc., or more other cells types) in the mixture of cells adhered to the substrate. According to this embodiment, the at least one other cell type detaches from the substrate at a lower detachment force. In embodiments, the cell of interest can then be detached from the substrate by the application of a higher detachment force. Alternatively, the cell of interest remains adhered to the substrate and can be cultured and/or can be subject to additional analysis, including for example, biochemical, protein marker, gene expression and/or genetic analysis.

In embodiments of the invention, the wall shear stress that results in 50% detachment of the cell type of interest (τ₅₀) is at least about 1.2, 1.5, 2, 3, 4, 5, 6, 7, 8, 9 or 10-fold higher as compared with the τ₅₀ for at least one other cell type in a mixture of cells. In embodiments, the wall shear stress that results in 50% detachment of the cell type of interest (τ₅₀) is less than about 70%, 60%, 50%, 40%, 30%, 20%, 10% or less as compared with the τ₅₀ for at least one other cell type in a mixture of cells.

The inventors have discovered that cancer-associated cells, and cells derived therefrom, have characteristic adhesive signatures that can be exploited to isolate such cells from each other (e.g., from other types of cancer-associated cells0 and from other cells adhered to a substrate (e.g., adherent cells in culture). For example, the methods of the invention find use in methods of isolating cancer-associated cells and/or cells derived therefrom, for example, to remove contaminating cells, to passage cells and/or to isolate rare cells, and the like. Accordingly, the methods of the invention can be practiced once (e.g., to identify a cell of interest) or two or more times (e.g., 2, 3, 4, 5, 6, 7, 8, 9, 10, etc., or more times; for example, in passaging cell cultures).

The at least one other cell type in the mixture of cells can comprise any other cell type that may be present in the mixture of cells, for example, as a contaminant (e.g., a cell that is not the cell of interest). In some embodiments, the other cell type can be a stem cell, a progenitor cell, a terminally differentiated cell, a stromal cell, an inflammatory cell, an explant cell and/or a progeny cell of any of these cells, as well as any other cell with a sufficient difference in adhesion strength to the substrate so that the cell of interest (e.g., a cancer-associated cell) can be selectively detached and isolated therefrom by an applied detachment force. In representative embodiments, the methods of the invention are used to isolate a cancer-associated cell subpopulation from a different subpopulation of cancer-associated cells, where the subpopulations of cancer-associated cells can be distinguished on the basis of adhesion strength to the substrate.

Any detachment force can be used that is sufficient to selectively detach the cell of interest (e.g., cancer-associated cell as compared with the at least one other cell type in a mixture of cells (e.g., a mixture of cultured cells) adherent to a substrate. In representative embodiments, the detachment force provides a wall shear stress in the range of about 20 or 50 to about 500 or 1500 dynes/cm². Other exemplary detachment forces are described herein.

In practicing the present invention, any two (or more, e.g., 3, 4, 5, 6, 7, 8, 9, 10, etc., or more) adherent cells (e.g., in culture) with sufficiently different adhesion strength to the substrate can be separated. In some embodiments, the two or more adherent cells can be from different cell types or lineages or different tumors and in some embodiments, the two or more adherent cells can be from the same cell type or lineage or tumor. In embodiments of the invention, the cell of interest detaches at a lower detachment force as compared with the at least one other cell type. Alternatively, the cell of interest can detach at a higher detachment force as compared with the at least one other cell type.

Cells isolated according to the methods of the invention are generally viable and/or retain the ability to divide and produce progeny cells. For example, in embodiments of the invention, at least about 50%, 60%, 70%, 80%, 90%, 95%, 96%, 97%, 98%, 99% or more of the cells are viable and/or retain the ability to divide and produce progeny cells.

Further, in embodiments of the invention, the cells are isolated with high efficiency and/or to a high level of purity. In embodiments of the invention, at least about 50%, 60%, 70%, 80%, 90%, 95%, 96%, 97%, 98%, 99% or more of the cells of interest in the mixture of animal cells adhered to the substrate are isolated. In embodiments, a plurality of the cells of interest are isolated with at least about 50%, 60%, 70%, 80%, 90%, 95%, 96%, 97%, 98%, 99% or more purity.

In addition, the isolation methods provided herein have been found to be quite robust and can isolate cells present at a wide range of starting concentrations in a mixture of cells. For example, in embodiments of the invention, the cell of interest constitutes less than about 50%, 40%, 30%, 25%, 20%, 15%, 10%, 5%, 4%, 3%, 2% or 1% or less of the cells in the mixture of animal cells. In embodiments, the cell of interest constitutes at least about 50%, 60% 70%, 80%, 90%, 95%, 96%, 97%, 98%, 99% or more of the cells in the mixture of animal cells.

Cells isolated according to the methods of the invention can be used for any purpose, e.g., further culture, and/or evaluation, for example, by flow cytometry, biochemical analysis, mammosphere formation assay (MFA), tumorsphere assay, marker expression assay, ALDH expression assay, migration assay, tumor formation assay, in vivo tumorigenesis assay and/or gene expression analysis, as well as any other suitable analysis or assay.

The detachment force used in the methods of this invention can be applied to the mixture of cells using any suitable method. As nonlimiting examples, the detachment force can be applied by hydrodynamic force, centrifugal force and/or magnetic force. In embodiments, the method of applying the detachment force does not involve labeling the cells with a detectable label and/or affinity reagent. In embodiments, the method of applying the detachment force can involve labeling the cells with a detectable label and/or affinity reagent.

The detachment force can be applied for any suitable period of time to achieve the desired level of detachment and isolation. In embodiments, the detachment force is applied for at least about 0.5, 1, 2, 3, 4, 5, 6, 7, 8, 9, 10, 12, 14, 16, 18, 20 minutes and/or less than about 5, 6, 7, 8, 9, 10, 12, 14, 16, 18, 20, 25, 30, 35, 40, 45, 50, 55, 60, 75, 90, 105, 110 or 120 minutes (including all combinations of lower and upper values as long as the lower limit is less than the upper limit). In representative embodiments, the time period is from about 2 to 20 minutes. In embodiments, the time period is from about 5 to 15 minutes.

In representative embodiments, the method is carried out in a fluid flow chamber or fluid flow device, including, e.g., a microfluidic device, a millifluidic device, and/or a spinning disk device. Spinning disk technology can be employed in the methods of this invention, which is an adhesive force measurement system wherein cells are attached to a substrate (e.g., a cover slip) and spun. This system samples a large range of applied shear forces (τ), which are radially linear (r). This system is also dependent on fluid density (ρ), rotational speed (ω), and fluid viscosity (μ).

In some embodiments of this invention, the source of the cultured animal cells is a cancer cell line. In some embodiments, the source of the cultured animal cells can be primary tumor tissue, tissue or cells obtained from a biopsy, explanted tissue, a xenograft tumor and the like.

In some embodiments of this invention, the cancer-associated cell is from a cancer selected from the group consisting of melanoma, adenocarcinoma, thymoma, lymphoma (e.g., non-Hodgkin's lymphoma, Hodgkin's lymphoma), sarcoma, lung cancer, liver cancer, colorectal cancer, leukemia, uterine cancer, breast cancer, prostate cancer, ovarian cancer, cervical cancer, vaginal cancer, vulvar cancer, bladder cancer, kidney cancer, pancreatic cancer, stomach cancer, esophageal cancer, brain cancer, central nervous system cancer, thyroid cancer, skin cancer, penile cancer, bile duct cancer, testicular cancer, paratesticular cancer, spleen cancer, vascular cancer, salivary gland cancer, cardiac cancer, odontogenic cancer, oral cancer, adrenal gland cancer, ocular cancer, throat cancer, thymus cancer, fallopian tube cancer, gallbladder cancer and any other cancer now know or later identified.

The invention further provides an isolated cell and isolated populations and cultures of cells produced by the methods of the invention.

The present invention is more particularly described in the following examples that are intended as illustrative only since numerous modifications and variations therein will be apparent to those skilled in the art.

### EXAMPLES

We recently established a platform technology, micro Stem cell High-Efficiency Adhesion-based Recovery (μSHEAR), to isolate human pluripotent stem cells and differentiated progeny based on differences in adhesive forces in a label-free, rapid (<10 min), and efficient (>95% purity, >95% yield) manner using microfluidics. This technology was developed to purify pluripotent stem cells and progeny, but preliminary data described herein support its capacity to enrich tumor cells, including tumor-initiating cells (TICs; also called cancer stem cells or cancer stem-like cells). Differences in adhesive force signatures that the sub-populations of cancer cells and TICs exhibit can be exploited to selectively purify them with high efficiency using μSHEAR. This technology provides a broadly applicable, easily implemented, and robust method to purify and characterize cancer cell sub-populations for basic studies of cancer heterogeneity and to maximize the quality and utility of samples derived from biospecimens for downstream diagnostic and prognostic analyses.

Tumors are heterogeneous masses of cells. The purpose of the process is to separate different sub-population of cancer cells, including cancer stem cells/TICs. Within a population of cancer cells, some adhere strongly to the substrate while others adhere more weakly. A microfluidic device is used to apply specific amounts of force to the cancer cells and detach them. The studies described herein demonstrate that the different cell fractions are composed of different sub-populations of cells.

The method of isolation and separation of cancer sub-populations and cancer stem cells can be used for cancer diagnostics to shed more light onto the unique compositions of a patient's tumor. This information will be helpful when tailored to more personalized therapies for patients. Currently markers are used to separate sub-populations of cancer cells from a tumor biopsy or to characterize the composition of a tumors. However, these labels are not always available or specific enough. The present invention provides a label-free approach that provides different applications and complements current diagnostic approaches.

### Example 1

### A Scalable, High-Throughput Platform for Stem Cell Expansion and Isolation

Current methods of stem cell purification are limited, non-standardized, and empirical, often relying on enzymes, probes, or skilled technicians in order to effectively achieve isolation. Moreover, purification methods are often invasive, non-scalable, and result in either low purity or low survival of the isolated cells. We report the development of a novel platform for stem cell expansion and isolation that allows for scalability and automation. The microfluidic platform, micro-Stem cell High-Efficiency Adhesion based Recovery [μSHEAR], relies on unique adhesion signature of cell populations to fractionate and purify them. Furthermore, cells can be cultured long-term in the system without affecting their proliferation, survival, or potency. This platform has the potential to greatly automate the expansion and purification steps of stem cell culture, something that will become increasingly important as stem cell research continues to move from an academic setting to an industrial one. The platform is also a closed system, which facilitates the acquisition of current good manufacturing practices (CGMP) certification down the line.

### μSHEAR Microfluidic Platform

Cell populations have unique adhesive signatures that vary depending on cell type/state. Devices were sterilized, coated with an extracellular matrix such as fibronectin, and cells were introduced. After 16-24 hr, cells were exposed to predetermined amounts of shear force and collected. Recovered cells were characterized for purity, survival, and potency.

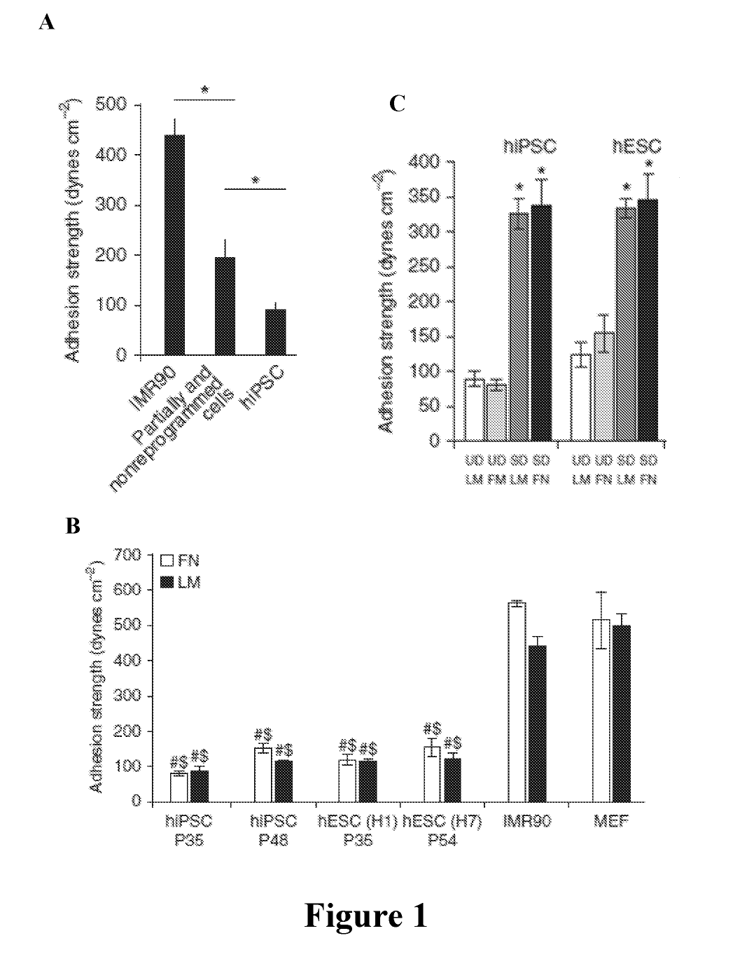

Adhesion signature strength of human induced pluripotent stem cells (hiPSCs) and human embryonic stem cells (hESs) undergoing reprogramming and differentiation is depicted in FIG. 1. Adhesion strength of cells during reprogramming is shown in FIG. 1, Panel A and the indicated cell types on fibronectin (FN) and laminin (LM) is shown in FIG. 1, Panel B. Adhesion strength for undifferentiated (UD) and spontaneously differentiating (SD) cultures of hiPSCs and hESCs on FN or LM is shown in FIG. 1, Panel C.

### Pluripotent Stem Cell Isolation and Expansion

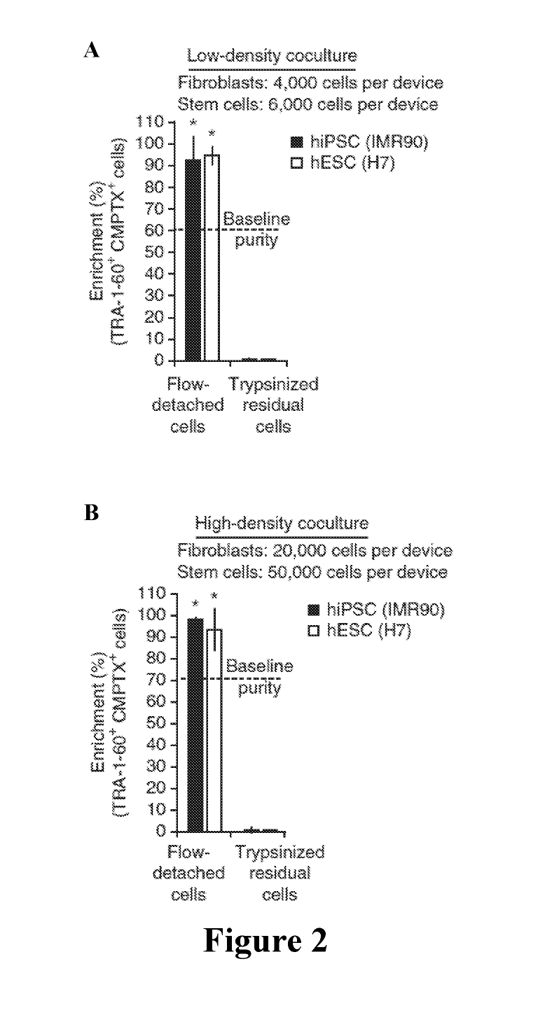

Adhesion strength-based isolation of pluripotent stem cells in microfluidic devices is depicted in FIG. 2. Selective isolation of hiPSCs was observed at a shear stress of 85-125 dynes cm⁻² when cocultured with IMR90 cells at low and high density. Enrichment of hiPSCs and hESCs isolated at 85-125 dynes cm⁻² from a coculture with IMR90 and mouse embryonic fibroblast cells are shown in FIG. 2, Panels A and B, respectively. Graphs show mean±s.d. (*P <0.05, n=3).

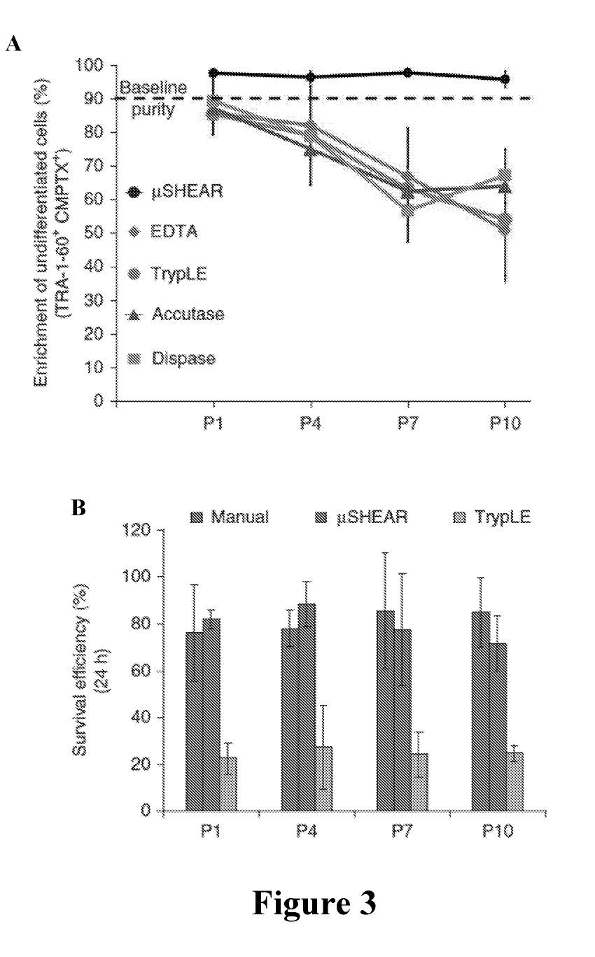

Continued culture and expansion of hiPSCs in μSHEAR platform is depicted in FIG. 3. The hiPSCs can be expanded within the microfluidic devices while maintaining equal or higher degrees of purity (FIG. 3, Panel A) and survival (FIG. 3, Panel B) than conventional methods of purification. Marker expression remained unchanged.

### Tumor Initialing Cell Isolation

The μSHEAR platform can have applications in the purification of TICs, which are cancer cells with increased tumorigenic capacity that drive cancer relapse and metastasis, as well as other sub-populations of cancer cells. To examine this, breast cancer cells were inserted into the microfluidic device and three fractions of cells were collected: a rinse fraction (R), a target shear stress fraction (TS), and the cells that remained in the device (RC). Recovered cells were seeded at a constant concentration into a Mammosphere Formation Assay (MFA)—this assay measures the in vitro ability of cells to form tumorspheres. Ten days post seeding, mammospheres were recovered and quantified. Cells in more strongly adhering fractions give rise to more and larger mammospheres.

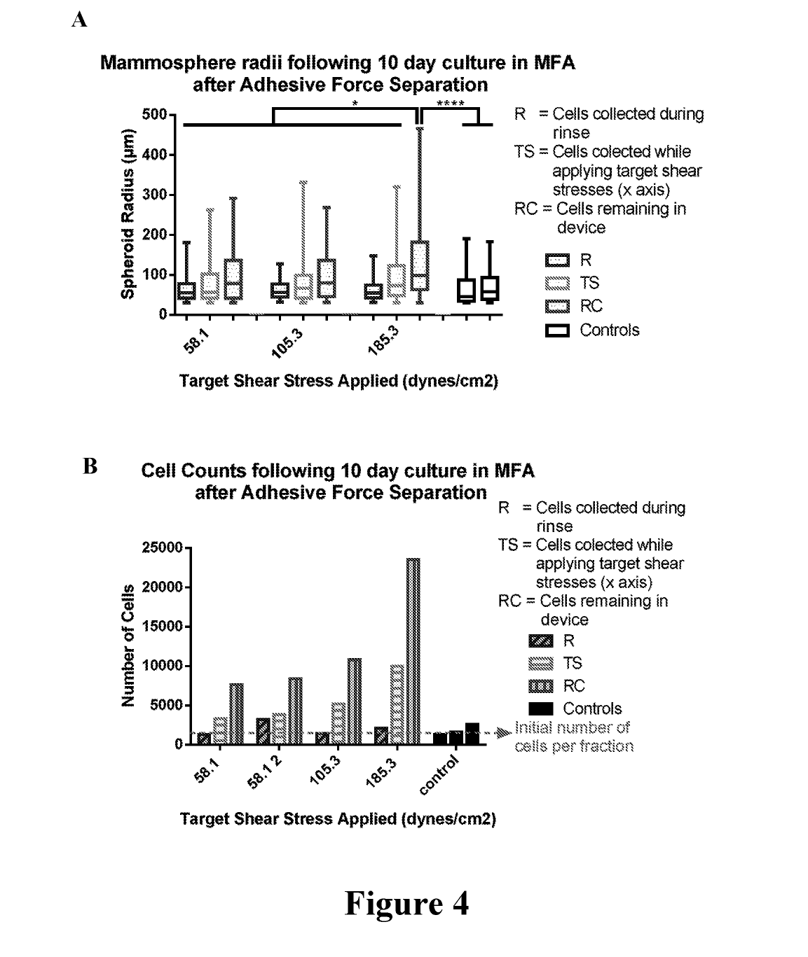

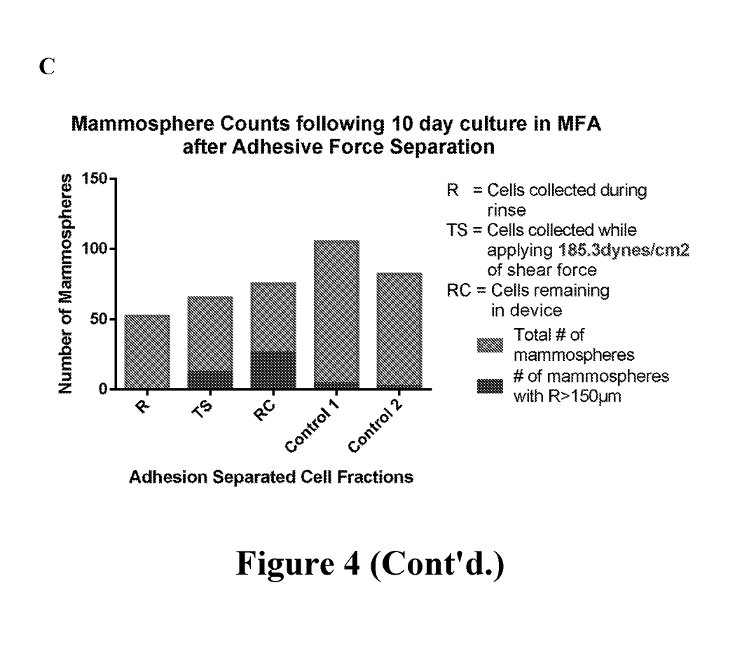

Cell counts and mammosphere characterization following 10 day MFA after adhesion force separation are depicted in FIG. 4. Cells that strongly attach to the matrix produce larger mammospheres after a 10 day MFA are shown in FIG. 4, Panel A. Mammospheres were disassociated into single cells and counted, as shown in FIG. 4, Panel B. The fraction of cells that attached the strongest (RC) displayed a 5-15 fold increase in the number of cells at the 10 day time point compared to the 0.8-1.7 fold increase seen in the controls. Furthermore, as the selection adhesive force for the RC fraction was increased, greater increases in the number of cells were seen. Mammosphere counts with 185.3 dynes cm⁻² of shear force used for separation, as shown in FIG. 4, Panel C. Cells that adhere more strongly to the matrix produce mammospheres with both a larger number of proliferative cells and a larger size.

### Conclusions

A) Cell populations have unique adhesion strength signatures that differ among them;

B) Differences in adhesion strengths among cells can be exploited by the μSHEAR platform in order to purify a fraction of them;

C) The μSHEAR platform can be used for extended culture of cells without negatively impacting them, which makes the platform a powerful tool for the automated and scalable expansion of stem cells;

D) The μSHEAR technology can have applications in the cancer field; and

E) Advantages of μSHEAR include its speed (10 min.), efficiency (95-99% purity, 99% survival), scalability, reproducibility, potential for automatization, and the fact that it is a closed system.

### Example 2

### Adhesive Signature Technology for Tumor Initiating Cell Purification in Cancer Research

Approximately 39.6% of people will be diagnosed with cancer. Cancer is responsible for 13% of all deaths worldwide. Cancers consist of heterogeneous populations of cells, which include tumor initiating cells (TICs), also called cancer stem cells (CSCs). TICs are a small subpopulation of cells that divide rapidly and are capable of establishing new tumors. TICs are responsible for cancer relapse and metastasis, and to date have been hard to target and purify. It is believed that TICs may have unique adhesive properties that differ from the adhesive properties of other cancer cells. Thus our objective is to apply a microfluidic platform to purify TICs from normal and cancer cells based on differences in adhesive forces.

### Adhesion Strength Measurements

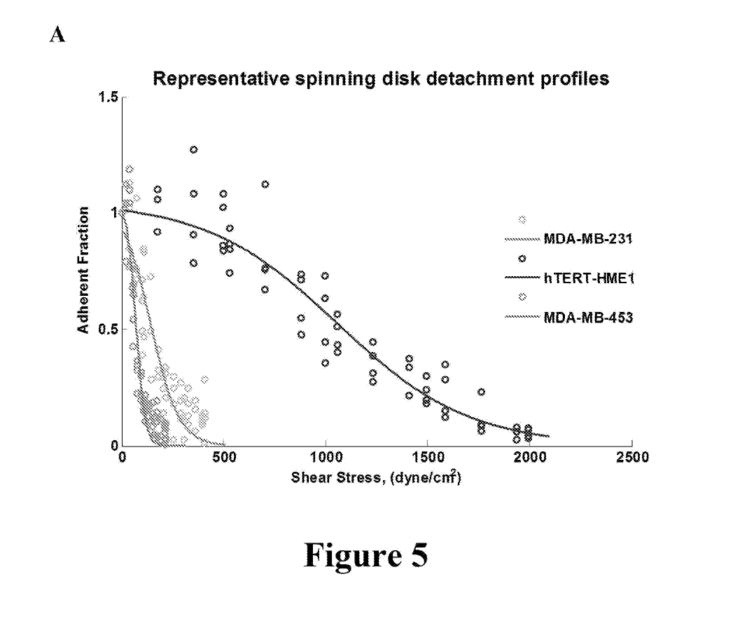

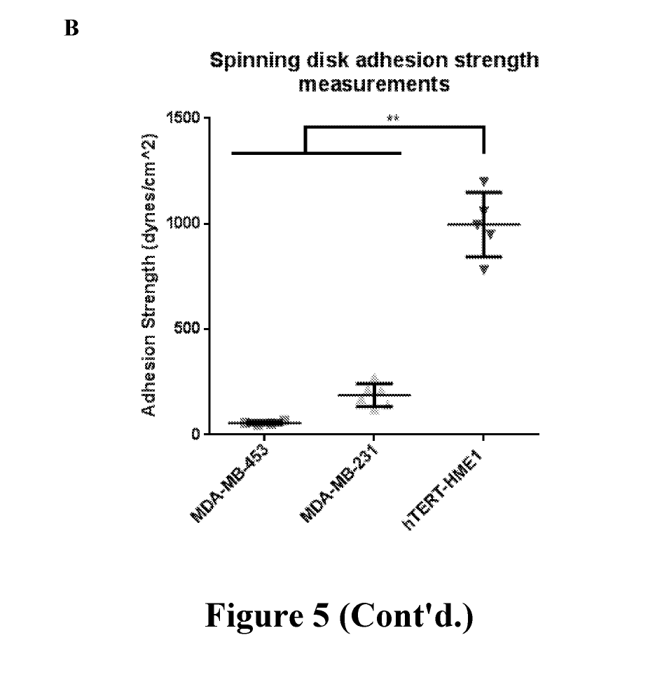

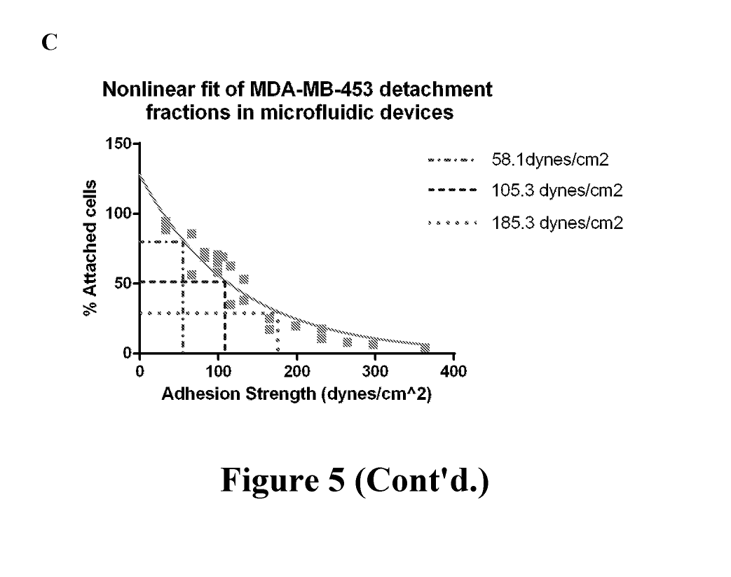

Adhesion strength of different cell populations is depicted in FIG. 5. Representative spinning disk detachment profiles are shown in FIG. 5, Panel A. Cells were grown on fibronectin-coated coverslips. After 24 hr, spinning disk experiments were performed and the adhesion strength was measured. A significant difference in adhesion is seen between immortalized hTERT-HME1 (non-cancer cells) and the MDA cancer lines as shown in FIG. 5, Panel B. Nonlinear fit of MDA-MB-453 detachment values after shear force application in microfluidic devices is shown in FIG. 5, Panel C. The shear force values used in the remaining experiments are highlighted.

### Microfluidic Experimental Platform

Cell populations have unique adhesive signatures that vary depending on cell type/state. μSHEAR devices were sterilized and coated with fibronectin and cancer cells were introduced. After 24 hr, cells were exposed to three predetermined amounts of shear force (58.1 dynes/cm², 105.3 dynes/cm², 185.3 dynes/cm²). For each condition, three fractions of cells were collected: a rinse fraction (R), a target shear stress fraction (values mentioned above, TS), and the cells that remained in the device (RC). Recovered cells were seeded at constant concentration into non-adherent wells and a Mammosphere Formation Assay (MFA) was performed. Ten days post seeding, mammospheres were recovered and quantified.

### Cell and Mammosphere Counts

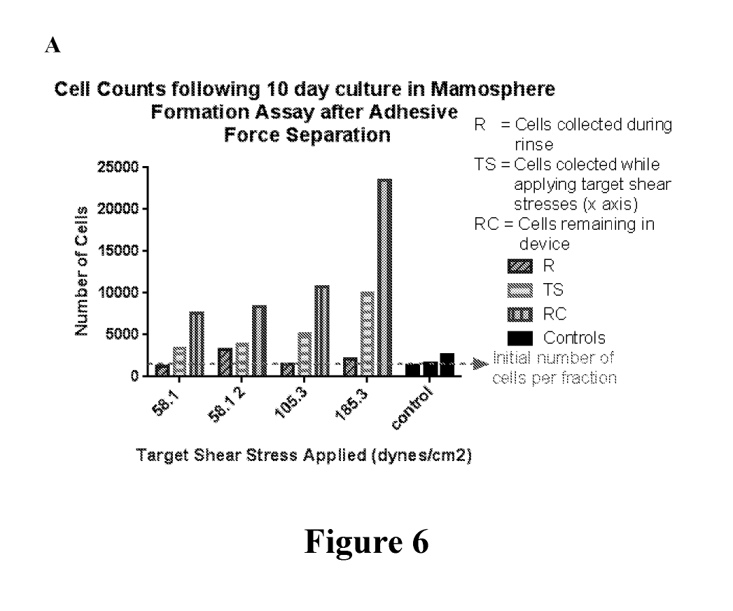

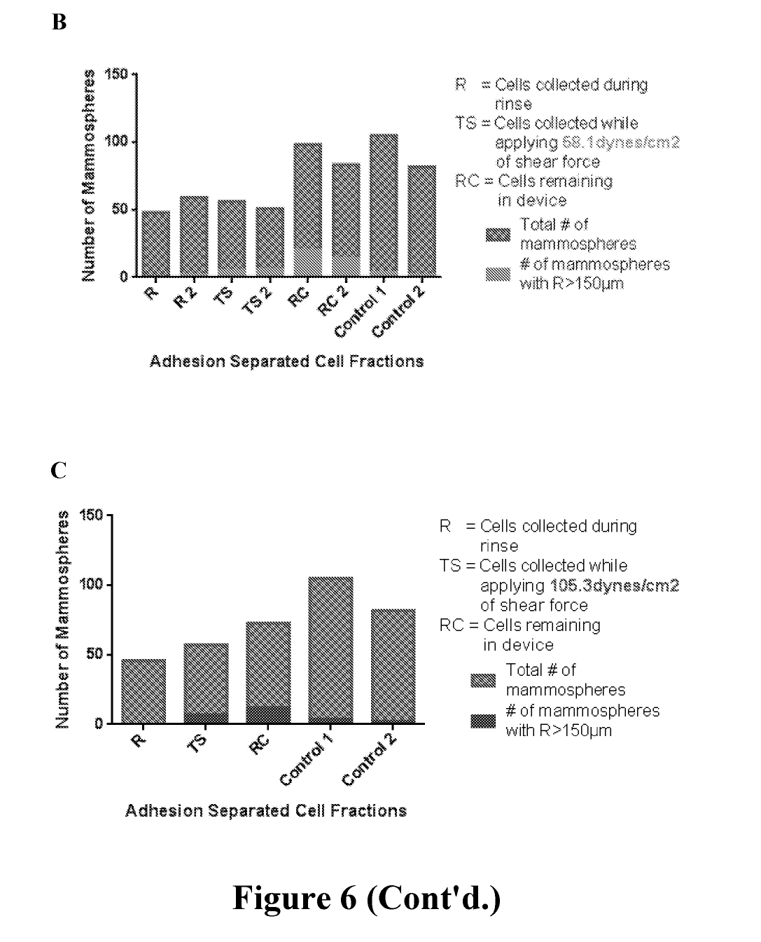

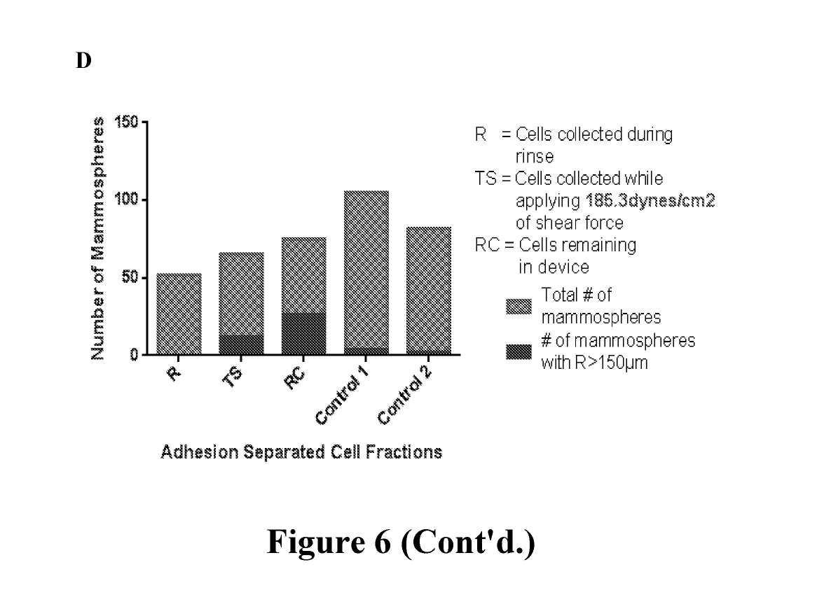

Cell and mammosphere counts following 10 day culture in MFA after adhesion force separation are depicted in FIG. 6. Mammospheres were disassociated into single cells and counted as shown in FIG. 6, Panel A. The fraction of cells that attached the strongest (RC) displayed a 5-15 fold increase in the number of cells at the 10 day time point compared to the 0.8-1.7 fold increase seen in the controls. Furthermore, as the selection adhesive force for the RC fraction was increased, greater increases in the number of cells were seen. Mammosphere counts with varying degrees of shear force used for purification are shown in FIG. 6, Panels B-D: 58.1 dynes/cm² (FIG. 6, Panel B), 105.3 dynes/cm² (FIG. 6, Panel C), and 185.3 dynes/cm² (FIG. 6, Panel D). Cells that adhere more strongly to the matrix produce mammospheres with both a larger number of proliferative cells and larger size.

### Mammosphere Size

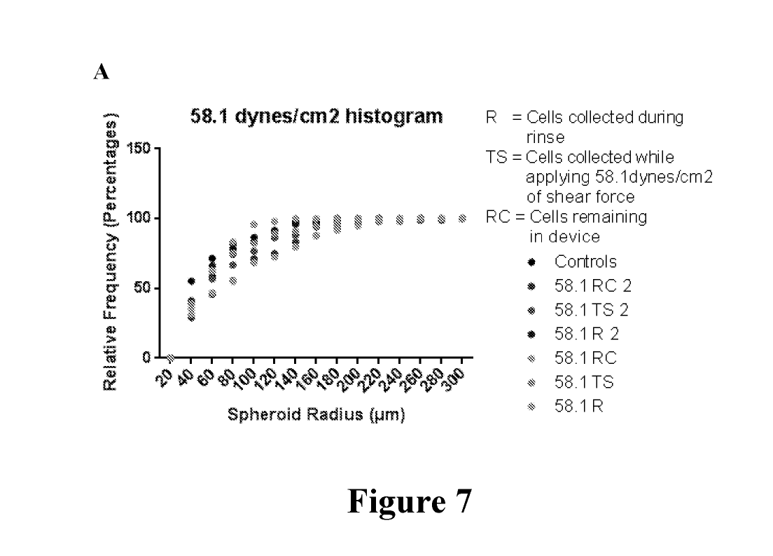

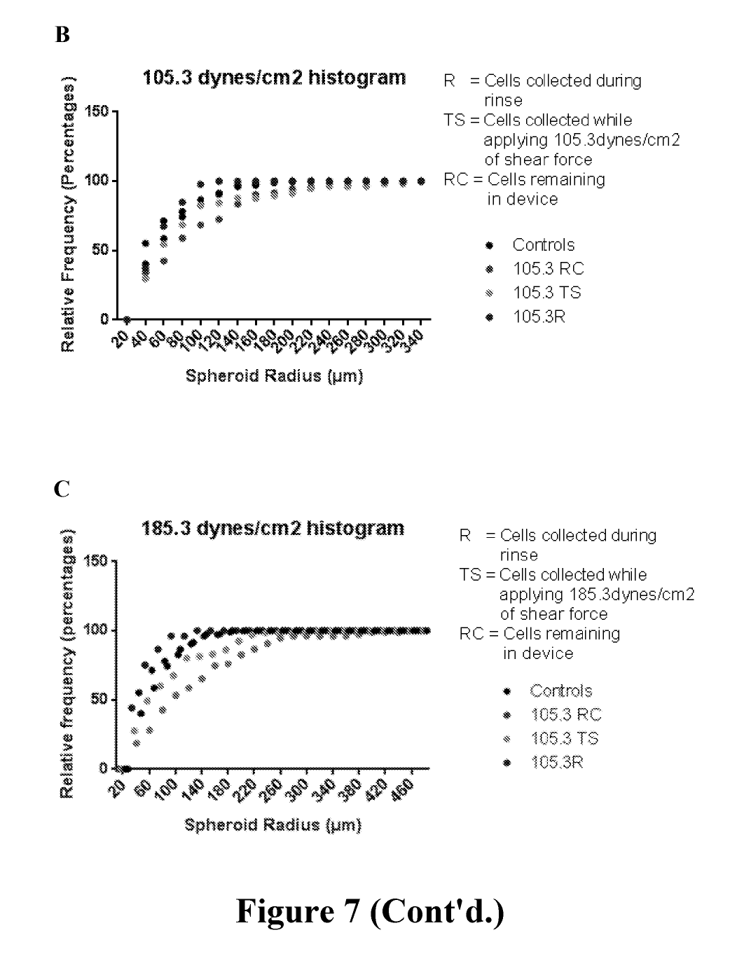

Quantification of mammosphere size is depicted in FIG. 7. FIG. 7, Panels A-C show histograms of the mammospheres' radii for cells separated with 58.1 dynes/cm² (FIG. 7, Panel A), 105.3 dynes/cm² (FIG. 7, Panel B), and 185.3 dynes/cm² (FIG. 7, Panel C). The probability distribution of the RC fraction in FIG. 7, Panel C is significantly different than the others.

Imaging of mammospheres in the control and the R, TS, and RC fractions following ten days in MFA after adhesion force separation with 185.3 dynes/cm² of shear showed that cells in more strongly adhering fractions give rise to more and larger mammospheres.

### Conclusions

A) Different cancer cell lines have distinct adhesive signatures that differ from non-cancerous cells as well as among cancer cell lines;

B) Cancer cells with a greater mammosphere formation potential can be separated via adhesion force-based separation; and

C) Strongly adhering cancer cells have a higher mammosphere formation capability.

### Example 3

### Adhesive Signature Technology for Cancer Cell Isolation

In spite of major therapeutic advances, cancer relapse and low rates of patient response persist. This failure is due in part to the heterogeneity within tumors and the differential responses to treatment that cancer cell subpopulations exhibit. The strength with which a cell adheres to the extracellular matrix is dependent on both the cell's lineage and state. The objective of this research is to investigate the adhesion properties of cancer cells and the differences therein. Using the spinning disk technology, a hydrodynamic assay that enables adhesion strength measurements, a panel of breast cancer cell lines was compared to the hTERT-HME1 non-cancerous immortalized breast cell line and a significant difference in adhesion properties was observed. Based on these results, a mouse tumor model was used to generate intradermal tumors in mice using the eGFP+B16 melanoma mouse cancer cell line. After ten days, tumors and the surrounded tissues were removed, digested into single cells, and introduced into μSHEAR microfluidic devices. After 24 hr, cells were exposed to shear force in order to isolate the eGFP+B16 cancerous cells from non-cancerous eGFP-cells. These data suggest that the cancerous cells can be enriched by use of adhesive force differences.

### Example 4

### Adhesive Signature Technology for Tumor Initiating Cell Purification in Cancer Research

In spite of major therapeutic advances, cancer relapse and low rates of patient response persist. This failure is due in part to a small subpopulation of tumor initiating cells (TICs) with stem cell like properties that are responsible for the growth of the tumor and the progression of metastasis. Currently, no efficient and reliable methods to isolate TICs for study exist. The objective of this research is to isolate the rare TICs from the general cancer cell population by exploiting differences in adhesion strength. To study these differences, we measured the adhesion strength (adhesive signature) of a panel of breast cancer cell lines to fibronectin using a hydrodynamic assay. Based on this screen, we selected the MDA-MB-231, MDA-MB-453, and MCF7 cell lines for purification of TICs using the μSHEAR technology. Briefly, microfluidic channels were sterilized and coated with fibronectin. MDA-MB-231, MDA-MB-453 or MCF7 breast cancer cells were enzymatically disassociated, pipetted into the inlet reservoir, and cultured in the device for 24 hr before detachment experiments. Cells were exposed to different shear forces to selectively detach cell sub-populations. Recovered cell/colonies were counted, seeded into a mammosphere formation assay (MFA), and analyzed after 10 days

The ability to form mammospheres is characteristic of TICs. After μSHEAR mediated separation of breast cancer cells into three fractions, the strongest adhering fraction consistently produced larger and more mammospheres. Furthermore, as the selection adhesive force for the adhered fraction was increased, greater increases in mammosphere number and size were observed. After 10 days, the mammospheres were disassociated and the number of cells quantified. A 5-15 fold increase in the final number of cells was observed in the strongly adherent fractions of cells, compared to 0.8-1.7 fold increase in the unsorted controls. These results show that cancer cells with a higher mammosphere formation potential, a hallmark of characteristic TICs, have a higher adhesion strength. Furthermore, these cells can be enriched via adhesion based separation giving rise to more and larger mammospheres than their less adherent counterparts. These results indicate that TICs could be separated based on adhesive forces.

### Example 5

### Adhesive Signature Technology for Tumor Initiating Cell Purification in Cancer Research

The objective of this research is to isolate the rare tumor initiating cells from the general cancer cell population by exploiting differences in adhesion strength.

Methods: The microfluidic channels were sterilized and coated with fibronectin. MDA-MB-231, MDA-MB-453 or MCF7 breast cancer cells were enzymatically disassociated, pipetted into the inlet reservoir, and cultured in the device for 24 hr before detachment experiments. Cells were exposed to fluid flow at predetermined PBS flow rates. Recovered cell/colonies were counted and seeded into a mammosphere formation assay (MFA). After 10 days, mammosphere number and size were quantified.

Results: The ability to form mammospheres is characteristic of TICs. After μSHEAR mediated separation of breast cancer cells into three fractions, the strongest adhering fraction consistently produced bigger and more mammospheres. Furthermore, as the selection adhesive force for the adhered fraction was increased, greater increases in mammosphere number and size were observed. After 10 days, the mammospheres were disassociated and the number of cells quantified. A 5-15 fold increase in the final number of cells was observed in the strongly adherent fractions of cells, compared to 0.8-1.7 fold increase in the unsorted controls.

Conclusions: These results show that cancer cells with a higher mammosphere formation potential, a hallmark of characteristic TICs, have a higher adhesion strength to the matrix. Furthermore, these cells can be enriched via adhesion based separation giving rise to more and larger mammospheres than their less adherent counterparts. Together, these results show that tumor initiating cells could be separated based on adhesion.

### Example 6

### Adhesion Signature Based Enrichment of Tumor Initiating Cells (TICs)

In spite of major therapeutic advances, cancer relapse and low rates of patient response to cancer therapeutics persist. This failure is due in part to a small subpopulation of tumor initiating cells (TICs) with stem cell-Eke properties that are responsible for the growth of the tumor and the progression of metastasis. These cells are capable of surviving chemotherapy, rendering them highly resistant to conventional cancer therapies. Although the question of whether TICs are stem cells remains a controversial topic in the cancer field, it has become increasingly evident that a better understanding of their biology and function is necessary to effectively treat cancer and eradicate tumors without allowing for relapse to occur.

This project aims to develop an objective, label-free, fast, and scalable method for TIC enrichment based on the adhesion strength signature of these cells. Currently, no efficient and reliable methods to isolate TICs exist. Although many in the field rely on surface marker expression profiles, these are variable and subjective, which hinders the study of TIC biology. The hypothesis is that subtypes of cancer cells may exhibit distinct ‘adhesive force signatures’ that can be exploited to selectively purify TICs and other cancer cell sub-populations with high efficiency using the μSHEAR technology. The significance of this work is the development of a novel platform for objective, reliable, and scalable TIC purification.

Tumor initiating cells (TICs), a subpopulation of cancerous cells within tumors responsible for their maintenance, present a major hurdle to cancer treatment and recovery because of their resistance to conventional therapies. While conventional cancer therapies target and often succeed in killing the bulk of the tumor's cancer cells, TICs, sometimes called cancer stem cells (CSC) or cancer stem-like cells (CSLCs), are resistant to these treatments, surviving hostile microenvironments and driving cancer relapse and metastasis. Unlike bulk cancer cells, TICs have the ability to self-renew and differentiate into many subtypes of cancer cells. TICs have been identified in a variety of cancer types in both primary tumors and cancer cell lines by use of surface marker expression profiles as well as the ability to form tumorspheres and xenograft tumors. Nevertheless, the surface marker expression profiles used to isolate TICs vary widely among cancer types and even within tumor samples and cell lines of the same cancer type. The inability to isolate TICs with high purity/efficiency has complicated the development of new therapies to specifically target these cells and continues to be a major hurdle in the cancer research and diagnostic fields.

Our lab has developed a technology to isolate cells based on their unique adhesion binding strength to the matrix. This novel technology (micro-Stem cell High-Efficiency Adhesion based Recovery μSHEARD consists of a microfluidic device that applies varying degrees of shear force to adherent cells. Using this device, human pluripotent stem cells (both human induced pluripotent stem cells [hiPSCs] and human embryonic stem cells [ESCs]) have been isolated from their parental cells, spontaneously differentiated cells, and partially reprogrammed cells with high reproducibility, yield (>97%), purity (95-99%), and survival (>95%) rates. The process is fast (<10 min), label free, and scalable. The objective of this project is to characterize the adhesion strength properties of TICs and exploit any differences to isolate them from the general cancer cell population.

We will study the adhesion properties of normal (hTERT-HME1) and cancer (MCF-7, MDA-MB-231, MDA-MB-453) mammary cell lines and cells established from primary human colonic biopsies by use of the hydrodynamic spinning disk technology and the μSHEAR technology. For each cell line, we will purify sub-populations at different adhesive force levels and evaluate their expression levels of established TIC markers (CD24, CD44, ESA, ALDH). Isolated sub-populations will be challenged using assays for tumor spheroid formation and agarose colony formation. Sub-populations of interest will then be implanted into NOD/SCID mice to examine their ability to form xenograft tumors. This study will demonstrate whether TIC sub-populations can be purified by differences in adhesive force signature and how the adhesive force signature correlates to surface marker profile and tumorigenicity.

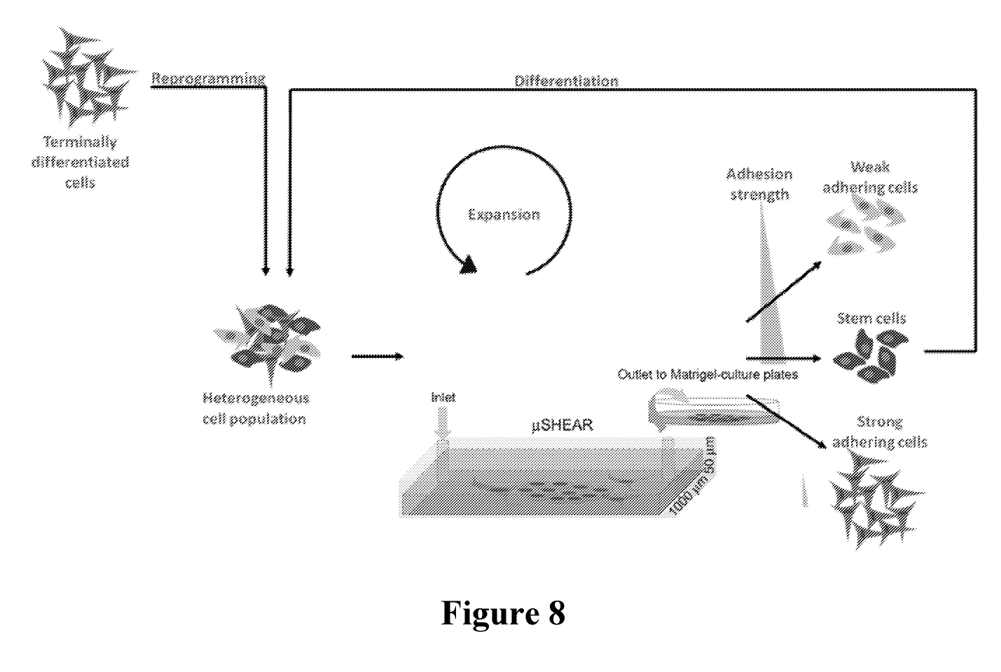

Xenograft tumors of human cancer cells (MCF-7, MDA-MB-231, MDA-MB-453, colonic tumor-forming cells) will be explanted, dissociated, purified and profiled using the microfluidic platform (FIG. 8). Purified sub-populations will then be assessed for tumorsphere and colony formation, invasiveness into Matrigel, and secondary tumor formation. This study will establish the ability of the integrated microfluidics platform to purify and identify TIC sub-populations from xenograft tumors.

The proposed project is innovative because it will use state of the art bioengineering technologies develop a novel method of TIC isolation from both cancer cells lines and xenograft tumors. Furthermore, this novel technology will provide an objective and label-free alternative to current TIC isolation approaches which will be fast, easy to use, and scalable. The methods developed have applications in cancer research by facilitating the study of TICs as well as clinically in cancer diagnostics and prognostics.

Tumors are heterogeneous tissues that contain many subpopulations of cells. Tumor initiating cells (TICs), a subpopulation of cancerous cells within tumors responsible for their maintenance, present a major hurdle to cancer treatment and recovery because of their resistance to conventional therapies. While conventional cancer therapies target and often succeed in killing the bulk of the tumor's cancer cells, TICs, sometimes called cancer stem cells (CSC) or cancer stem-like cells (CSLCs), prove to be resistant to these therapies, surviving hostile microenvironments and driving cancer relapse and metastasis. Unlike bulk cancer cells, TICs have the ability to self-renew and differentiate into many subtypes of cancer cells.

TIC-enriched populations have been identified in established cell lines and patient samples using a variety of techniques including discrete surface markers (CD44hi/CD241o, CD133+, ALDH+, ESA+) and their ability to generate tumorspheres and xenograft tumors. Although surface marker expression is the most widely used method for TIC isolation, the expression profiles vary widely among cancer of different tissue origin and moreover, among TIC populations of different tumors and cell lines within a specific tissue. To date, there is no universal marker profile to identify TICs. The inability to effectively, scalably, and objectively purify TIC subpopulations is a profound impediment to characterizing the biology of these cells with precision as well as analyzing patient samples for effective diagnosis or prognosis. Therefore, there is a significant and unmet need for unbiased, efficient, label-free technologies for the identification and purification of various cancer cell populations from heterogeneous cultures and tumors.

Increasing numbers of parallels are being drawn between cancer and stem cell research. Until recently, cancer progression was described using mainly the clonal evolution model, which postulates that cancers evolve by a repeating process of clonal expansion, mutation, and selection. As cancer progresses, different mutations accumulate in clones within the tumor and selective pressure leads to the survival of some clones and the extinction of others in a manner similar to Darwinian natural selection. Within this model, all cancer cells have the ability to rapidly divide and give rise to a new tumor. A growing body of data supports an alternative view of cancer, dubbed the cancer stem cell (CSC) model. In contrast to the clonal evolution model, the CSC model proposes a hierarchical organization of cells in which a small population of tumor-initiating cells (TICs) are capable of self-renewal into more TICs and ‘differentiation’ into bulk cancer cells. As the name suggests, TICs are defined by their unique ability to initiate new tumors, whereas other cancer cells cannot, but also display distinct marker expression profiles, chemotherapy/drug resistance, and biophysical properties (Table 1).

TICs are thought to be responsible for the maintenance, progression, recurrence, and metastasis of cancer. Often, their higher propensity to be drug-resistant allows TICs to survive conventional therapies and leads to drug resistant cancer relapse and metastasis development. TICs are usually rare populations within a tumor and their purification has proven challenging, even after in vitro culture. Efficient isolation and enrichment of TICs would facilitate their study and the development of drugs that selectively target them.

It is important to distinguish between the cancer cell of origin (CCO) that initiates a tumor and the CSCs/TICs that sustain it, as they may not necessarily be related. The CCO is the original cell that accumulates the first genetic mutations that lead to cancer. While the CCO is involved in the initiation of the primary tumor, CSCs/TICs are involved in the maintenance of this tumor and the initiation of secondary ones. The terms CSC and TIC are often used interchangeably to denote cancer cells that can self-renew to make more of themselves as well as ‘differentiate’ into bulk cancer cells. As mentioned previously, these cells are often referred to as cancer stern cells because of the similarities to somatic stem cells and tumor initiating cells because they are able to initiate tumors in immunocompromised mice.

Many different methods of TIC purification have been developed to exploit unique attributes in these cells. Common methods of enrichment include surface marker-based purification and isolation based on TIC intrinsic functional markers, such as ALDH expression, reactive oxygen species (ROS) levels, flow cytometric side population (SP) analysis, and mitochondrial membrane potential differences. Many of these purification platforms rely on probes such as antibodies and separation technologies such as flow cytometry and magnetic beads. These methods have several drawbacks including high price, non-specificity, inability to scale-up, and lack of robustness.

TICs have been identified in many types of solid tumors based on their expression of surface markers (Table 2). Various surface markers continue to be identified; however, no universal marker exists. Instead, TIC surface markers appear to be tissue specific and may vary among different tumors requiring extensive validation. Moreover, even well validated markers such as CD133 seem to fail to specifically identify TICs in certain applications. In spite of their limitations, surface markers are widely used for TIC purification, with some groups developing non-antibody based aptamer probes. Many of the developed markers are conjugated with fluorescence labels and used in combination with techniques such as fluorescence-activated cell sorting (FACS) and magnetic-activated cell sorting (MACS) for isolation.

### Adhesion Signature Force Measurement of Breast Cancer Cell Lines

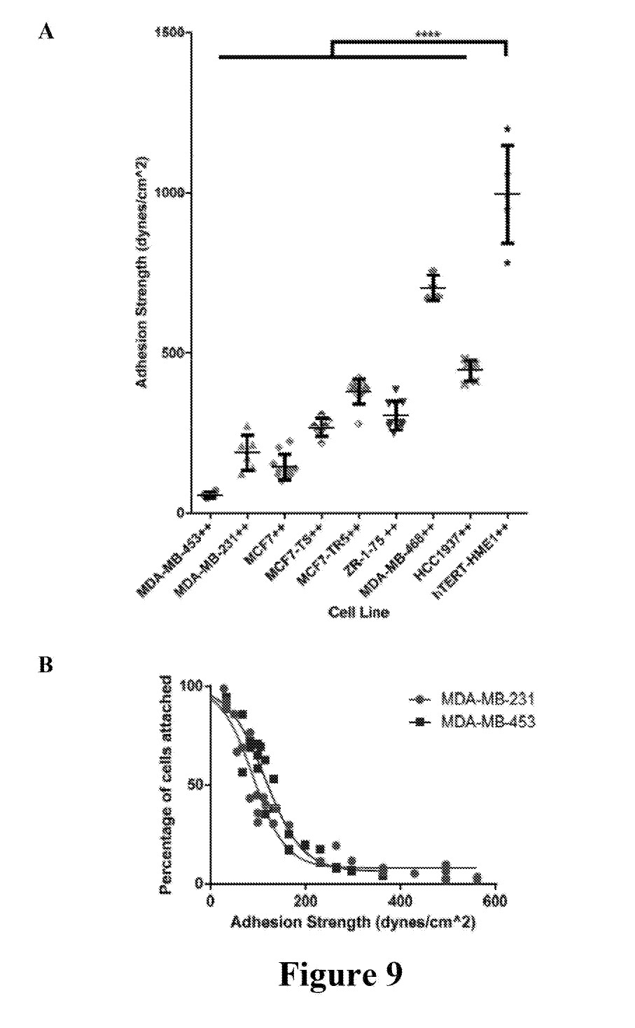

The adhesion signature force of a panel of breast cancer cell lines was measured using the spinning disk technology. Circular cover slips (25 mm diameter) were sterilized with ethanol, coated with fibronectin (10 μg/mL) for 30 min, and blocked with a 1% solution of bovine serum albumin (BSA) for 30 min. Cells were seeded onto fibronectin-coated circular coverslips and cultured overnight at concentrations of 75,000-200,000 cells/mL depending on the cell line in order to achieve 40-50% confluency. After 24 hr, the coverslips were spun for 5 min in phosphate buffered saline solution buffer (PBS), thus applying a range of forces to the cells proportional to the cell's radial position in the cover slip. The cells were fixed with 4% paraformaldehyde for 15 min, permeabilized in a 0.05% Triton-X100 solution for 40 min, stained with DAPI for 30 min, washed three times with PBS, and mounted into slides for imaging. The number of cells at defined radial positions were then quantified by use of a fluorescence microscope with a mechanical stage. After fitting the data into sigmoidal curves, the τ₅₀ (force required to detach 50% of the cells) was calculated (FIG. 5, Panel A). Non-cancerous immortalized mammary cells (hTERT-HME1) had a significantly higher adhesion strength signature than all cancerous cell lines. Representative fits for hTERT-HME1, MDA-MB-231, and MDA-MD-453 cell lines are shown in FIG. 9, Panel A.

MDA-MB-231 and MDA-MD-453 cells were also stained fluorescently with CellTracker Red CMTPX, introduced into fibronectin-coated μSHEAR microfluidic devices (45 min coating with 10 μg/mL fibronectin followed by 45 min blocking with 1% BSA) and cultured overnight to permit cell adhesion. After 24 hr, the cells were exposed to well-defined shear forces controlled by a syringe pimp and their detachment monitored on a fluorescence microscope. The detachment fraction of the cells was calculated by dividing the number of cells remaining in the device after shear force application by the total number of cells present in the device before any force was applied. It is important to note that this approach allows for the live monitoring of the detachment of the cells, something that is not possible with the spinning disk technology. FIG. 9, Panel B shows the sigmoidal best fit detachment profiles for both cell lines. While >80% of the cells detach with 200 dynes/cm² of shear force, some remain attached in spite of much higher forces, suggesting the existence of a subpopulation of strongly adherent cells within the cell lines.

### μSHEAR Mediated Enrichment of TIC in MDA-MB-453 and MFA Characterization.

MDA-MB-453 cells were introduced into the μSHEAR microfluidic devices and cultured overnight to permit cell adhesion. Devices were first sterilized with ethanol, washed with PBS, coated with fibronectin (10 μg/mL in PBS, 45 min), and blocked with bovine serum albumin (1% BSA in PBS, 45 min). Cells were then introduced at a concentration of 10⁷ cells/mL and cultured at 37° C., 5% CO₂ overnight. The following day, predetermined amounts of force were applied to the cells for a 10 min period by flowing PBS at well-defined flow rates controlled by a syringe pump. After 10 min, the cells that remained attached were trypsinized for 5 min, 1 mL of Dulbecco's Modified Eagle Medium supplemented with 10% fetal bovine serum (DMEM, 10%FBS) was added to inactivate the trypsin, and the cells were collected along with those that detached. The two fractions of cells were centrifuged, counted, and 2,000 cells were seeded into each Corning Ultra Low Adhesion well of the mammosphere formation assay (MFA) assay which contained 2.0 mL of serum free media (DMEM, 10 ng/mL bFGF, 20 ng/mL EGF, 1× B27 supplement, 1% L-Glutamine, 10 μg/mL heparin, 0.5% methyl cellulose).

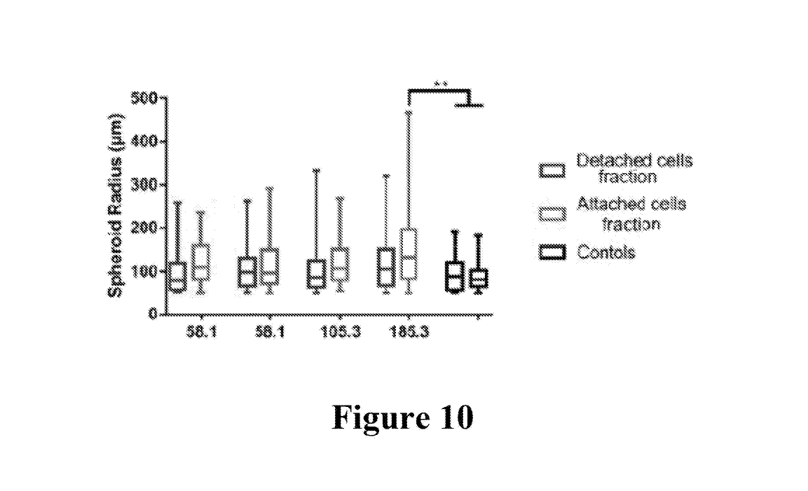

After 10 days of culture, the mammospheres were stained with Calcein-AM (1 μM) for 15 min and their radius was assessed using fluorescence microscopy. A mechanical stage was used to image the entire surface of the well and image the fluorescent mammospheres. The images taken were analyzed using an ImageJ macro and their radii assessed. The fraction of cells that remained attached to the device after the application of the highest degree of shear force had significantly larger mammospheres than control wells containing cells that had not been fractioned in the μSHEAR device (FIG. 10). The mammospheres were then mechanically disassociated by use of a Pipetman and the number of cells per well was counted using a Coulter counter (FIG. 6, Panel A).

### Xenograft Tumor Generation

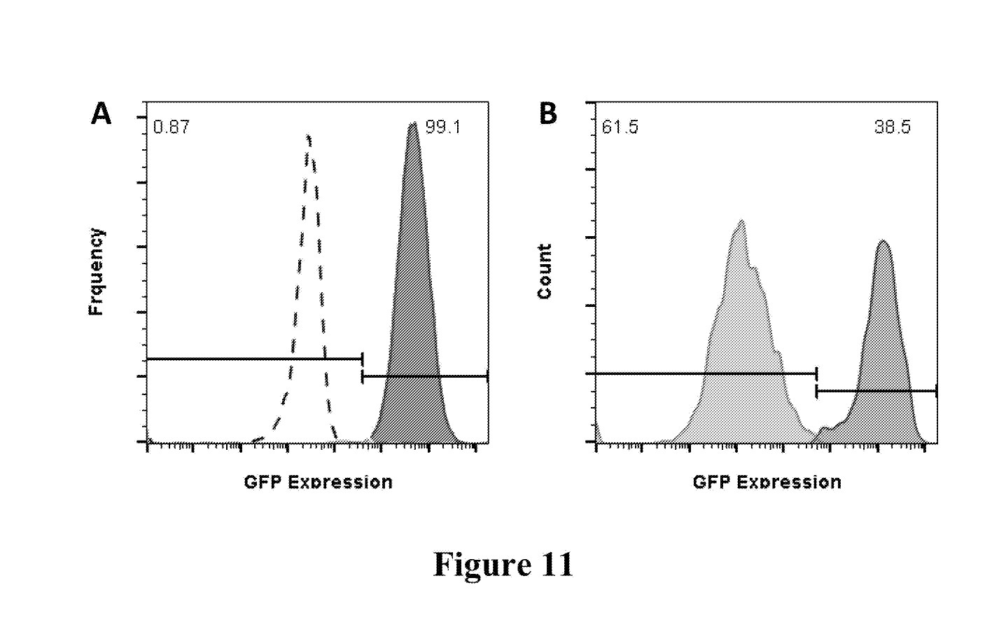

Stably transduced eGFP-B16 cells were generated by infecting B16 mouse melanoma cells with LV-CMV-eGFP lentivirus and sorting for eGFP+ cells on an Aria sorter. Briefly, cells were seeded at 60% confluency in 6-wells and allowed to attach overnight. The cultures were then lentivirally infected at several multiplicity of infections (MOIs) ranging from 10 to 50. Four days post-infection, the cells were trypsinized and the enhanced green fluorescent protein (eGFP) expression levels assessed in a flow cytometer. The MOI that gave the best efficiency of infection, MO1=50, was selected for further use. Two rounds of sorting were performed in order to achieve a >99% eGFP+ population of cells. eGFP B16 cells were injected (1×10⁶ cells in 30 μL Matrigel) intradermally into NOD/SCID mice to establish xenograft tumors. The tumors were excised after 10 days and digested enzymatically with collagenase D (0.375 U/mL) and hyalurodinase (125 U/mL) for 2 hr at 37° C., a 1:1 mixture of dispase (1 U/mL) and 0.25% trypsin-EDTA (5 min, 37° C.), red blood cell lysis buffer (5 min, room temperature), and DNAse (10 min, 0.1 mg/mL, room temp). The mixture of cells was filtered through a 40 μm filter twice. Flow cytometry and propidium iodide staining (15 min, 4° C., 1 μg/mL) were used to assess the viability of the recovered cells. eGFP+cells isolated from the tumor and eGFP-cells isolated from the surrounding tumor remained alive after the procedure (FIG. 11) and could successfully be introduced into the μSHEAR device.

Establishing the Ability of the μSHEAR Microfluidics Platform to Purify TIC Sub-Populations from Normal and Cancer Cell Lines Based on Adhesive Force Signatures.

Adhesion molecules, including integrins and FAK, are often dysregulated in cancer, contributing to disease progression and metastasis. Preliminary results using the spinning disk technology indicate that the adhesive signature strength with which breast cancer cells bind to fibronectin (FN) varies significantly among breast cancer cell lines and is significantly lower in cancerous cells relative to non-cancerous immortalized mammary cell lines (HME1-hTERT and MCF10A). Interestingly, even within a single cell line there appear to be subpopulations of cells that bind to FN with different amounts of force. These studies will investigate the possibility of exploiting these adhesive differences to purify subpopulations of cancerous cells, specifically tumor initiating cells (TICs). We hypothesize that TICs may have unique adhesive strength signatures. Furthermore, we hypothesize that we may be able to exploit these unique adhesive signatures to purify TICS from several cell lines (MCF7, MDA-MB-231, MDA-MD-453, and cells established from primary human biopsies) by using the μSHEAR technology.

### Adhesive Properties of Cancerous and Non-Cancerous Cell Lines.

The adhesive properties of normal (hTERT-HME I) and cancer (MCF-7, MDA-MB-231, MDA-MB-453) mammary cell lines as well as cells established from primary human colonic biopsies will be examined by use of the hydrodynamic spinning disk technology. Glass coverslips will be sterilized with ethanol, coated with fibronectin (10 μg/mL) for 30 min, blocked with 1% BSA for 30 min, and cells will be seeded onto them at concentrations ranging from 75,000-200,000 cells/mL in order to achieve 40-50% confluency. After 24 hr, the spinning disk device will be used to apply a range of forces to the cells proportional to the radial distance between the center of the coverslip and the position of the cell. Cells will be fixed with 4% paraformaldehyde for 15 min, permeabilized in a 0.05% triton solution for 40 min, stained with DAPI for 30 min, washed three times with PBS, and mounted into slides for imaging. The resulting cell detachment will be quantified and the τ₅₀, the amount of shear stress required to detach 50% of the cells, calculated.

### μSHEAR-Based Sub Population Separation and Evaluation of Their Tumorigenic Potential.

The μSHEAR technology will be used to separate subpopulations of cancer cells from the cell lines outlined above. The device will be coated with a saturating monolayer of human fibronectin to generate a well-defined cell adhesive substrate. To do so, devices will be first sterilized with ethanol, washed with PBS, coated with fibronectin (10 μg/mL in PBS, 45 min), and blocked with bovine serum albumin (1% BSA in PBS, 45 min). Cells are then introduced at a concentration of 10⁷ cells/mL and cultured at 37° C., 5% CO₂ overnight. The following day, specific amounts of force will be applied for a 10 min period by flowing PBS at well controlled flow rates controlled by a syringe pump. The hydrodynamic force applied on the cells will be increased to sample a range of shear forces and both the cells that detach and those that remain attached will be collected in two separate fractions for further characterization.

The collected cell fractions will then be analyzed for TIC phenotype. They will be stained with TIC markers such as CD44/CD22, CD133, and ESA using fluorescently labeled antibodies and ALHD with ALDEFLUOR reagent (StemCell) and analyzed using the flow cytometer. Isolated subpopulations will also be tested for tumorsphere formation using a mammosphere or colonosphere formation assay (MFA/CFA). The cells will be seeded at specific cell densities into Ultra-Low Adhesion plates (Corning) in media supplemented with methylcellulose and allowed to grow for ten days. To quantify the extent of spheroid formation, we will either use lentiviral transduced eGFP-expressing cells or we will stain the culture using Calcein-AM. The invasiveness of the cells will also be addressed using a Matrigel invasion assay (Chemicon).

Based on the results from the previous assays, we will select the hydrodynamic shear force that results in the largest enrichment of the TIC subpopulation and will implant the separated cells into NOD/SCID mice to examine their ability to form xenograft tumors. As a control, we will enrich for TICs in a cell sorter using either surface marker antibody mediated selection or ALDH expression level based separation. Cells in Matrigel (10³ or 10⁵ cells in 30 μL) will be injected subcutaneously into the dorsal flank of NOD/SCID mice; two cell doses will be used to examine tumorigenic potency. We will deliver bilateral cell injections to each mouse and use 4 mice per cell subpopulation. Tumor formation frequency and tumor volume at 21 days will be quantified in a blinded fashion to ensure scientific quality and integrity.

The TIC niche plays a crucial role in vivo in supporting TIC function, maintenance, and self-renewal capabilities. It is therefore crucial to recreate the niche-TIC interaction when studying the adhesion properties of TICs to achieve clinical relevance. We plan to produce xenograft tumors in NOD/SCID mice to attempt to separate cancer cells from non-cancerous surrounding cells as well as TICs from other cells. The adhesion signature differences among non-cancerous surrounding cells, non-TIC cancerous cells, and TICs may be large enough to enable their separation and enrichment by use of the μSHEAR microfluidic technology.

### Xenograft Tumor Model Generation.

We will examine whether TIC subpopulations can be isolated from xenograft tumors via differences in adhesive force using μSHEAR. Mouse cancer cells (B16) and human cancer cells (MCF7, MDA-MB231, MDA-MB453, CRC) will be injected (1×10⁶ cells in 30 μL Matrigel) subcutaneously into the dorsal flank of NOD/SCID mice to establish xenograft tumors. Because these cell lines contain various TIC subpopulations, the xenograft tumors are expected to be heterogeneous. We will analyze 6-8 tumors per cell line depending on the variance of the data obtained. At 11 days, tumors will be explanted and cells will be isolated by enzymatic and mechanical dissociation. They will be digested using a collagenase D/Hyalurodinase/Trypsin/Dispase cocktail.

### μSHEAR-Mediated Separation of Tumor Cells.

The μSHEAR devices will be coated with a saturated monolayer of human fibronectin to generate a well-defined cell adhesive substrate. Cells will be cultured in media within the microfluidic devices overnight and a specific amount of force will be applied for a 10 min period by flowing phosphate buffered saline solution at well controlled flow rates controlled by a syringe pump. The results from experiments described herein will be used to determine the target flow rates for TIC isolation. Both the detached cells and the cells that remain in the device will be collected for further functional characterization by use of the MFA/CFA assays described herein. The number and size of the spheroids will be analyzed as described herein as well as the cell's abilities to form secondary spheroids when dissociated into singly cells and cultured in suspension conditions.

### In Vitro Characterization of Cell Fractions.

Cells will be stained for the TIC markers CD24, CD44, ESA, and CD133 using fluorescently labeled antibodies or ALHD with ALDEFLUOR reagent and analyzed by flow cytometry. Cell populations will then be functionally characterized for invasiveness and colony formation by using a Matrigel invasion assay and the colony formation assay described herein. Based on these devices, cells will be isolated by using the μSHEAR technology and implanted into NOD/SCID mice to examine the ability of these tumor derived cells to form secondary tumors.

### Example 7

### μSHEAR Technology for Cancer Stem Cell Purification

Human stem cells represent disruptive technologies for the generation of (i) auto- and allo-genic cell sources for countless therapeutic applications and (ii) novel models for the study of human development and disease. Despite considerable progress in the identification of stem cell markers, development of culture conditions that maintain self-renewal capacity and direct differentiation, and genomic/proteomic analyses, there is a significant and unmet need for unbiased, efficient, label-free technologies for the purification of various stem cell populations such as parental/support cells, undifferentiated stem cells, partially committed/differentiated precursors, and differentiated progeny. This crucial need for robust purification technologies is relevant to adult (e.g., mesenchymal stem cells (MSC) and endothelial progenitor cells), embryonic (ES), and induced pluripotent (iPS) stem cells. Similarly, the field of cancer stem cells (CSC) also necessitates efficient platforms to purify these stem cells and their progeny at various stages of differentiation.

We recently established a platform technology, Micro Stem cell High-Efficiency Adhesion-based Recovery (μSHEAR), to isolate ES and iPS cells and differentiated progeny based on differences in adhesive forces in a label-free, rapid (<10 min), and efficient (>95% purity, >95% yield) manner using microfluidics. The objective of this project is to establish the broad application of μSHEAR to stem cell technologies as it relates to the purification of (i) adult MSC and CSC, and (ii) subpopulations at various stages of reprogramming in iPS cells. The central hypothesis is that specific populations of adult, iPS, and cancer stem cells may exhibit distinct ‘adhesive force signatures’ that can be exploited to selectively purify them with high efficiency using μSHEAR.

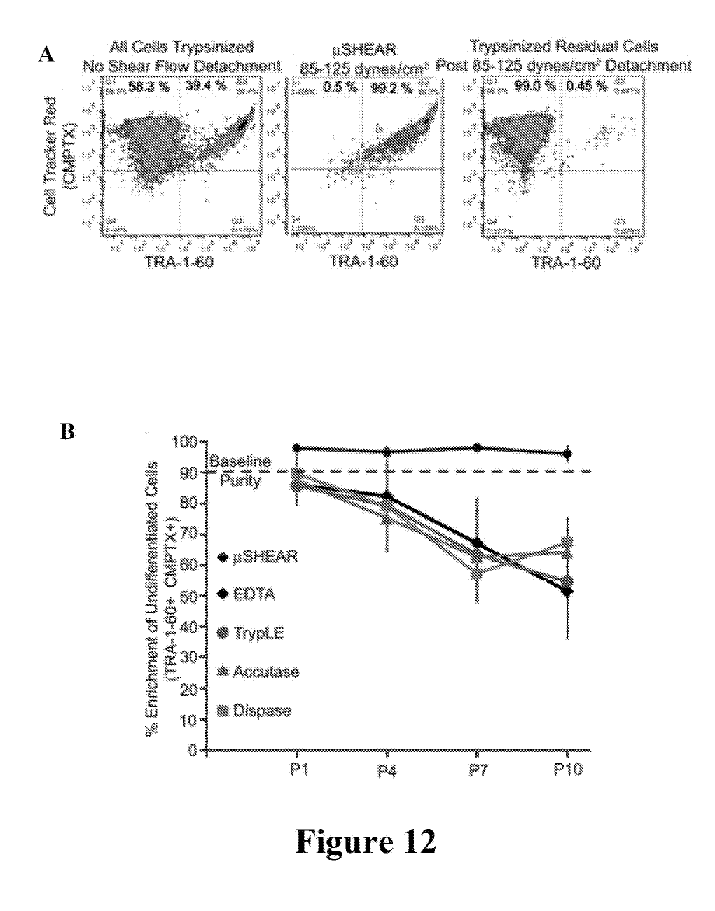

We exploited the unique adhesive signatures among pre- and post-reprogrammed states of hiPSC to develop a novel strategy to isolate and enrich for hiPSC from a heterogeneous population of cells during reprogramming. The distinct ‘adhesive signature’ of hiPSC was exploited to rapidly (<10 min) and efficiently isolate undifferentiated, bona fide hiPSC as intact colonies from parental fibroblasts, partially reprogrammed cells, and spontaneously differentiated (SD)-hiPSC via controlled fluid forces using microfluidics (FIG. 12, Panel A). We termed this technology μSHEAR (micro Stem cell High-Efficiency Adhesion-based Recovery). Undifferentiated hiPSC were isolated in a label-free fashion and enriched to >98% TRA-1-60-positive hiPSC population compared to the initial low purity co-culture (30% hiPSC). Quantitative analysis of the recovered live hiPSC with >97% purity (TRA-1-60+), irrespective of the levels of contaminating cells (6-70%), indicate that μSHEAR is a robust method to enrich undifferentiated cells from contaminating cell types. μSHEAR-based isolation resulted in repeated high purity (>97%) across 10 passages each 5-7 days apart, starting with a low 10% spontaneously differentiated population. In contrast, four routinely used solution or enzymatic passaging approaches (EDTA, TrypLE, Accutase, and Dispase) failed to selectively enrich undifferentiated cells and levels of spontaneous differentiation continuously increased over repeated passaging (FIG. 12, Panel B).

When collected and cultured on Matrigel-coated plates in ROCK inhibitor-supplemented mTeSR®1 media, μSHEAR-purified hiPSC appeared as undifferentiated colonies with no signs of differentiation even after 10 repeated detachments over 70 days. The recovered undifferentiated colonies retained their self renewal capacity and pluripotency as evidenced by OCT4 and SSEA4 expression at different passages, and differentiated into all three primary germ layers. Detailed gene expression analysis on μSHEAR vs. manually passaged hiPSC showed that the expression profiles of genes involved in maintaining sternness, self-renewal, pluripotency, and related growth factors were overall similar at passage 10 to those at passage 0, independent of passaging method. The expression profiles for differentiation and lineage-specific genes were either equivalent or down-regulated for both μSHEAR- and manual passaged hiPSC compared to the starting cells. Karyotype analysis demonstrated that μSHEAR passaged hiPSC exposed to 10 rounds of passaging exhibited no chromosomal abnormalities.

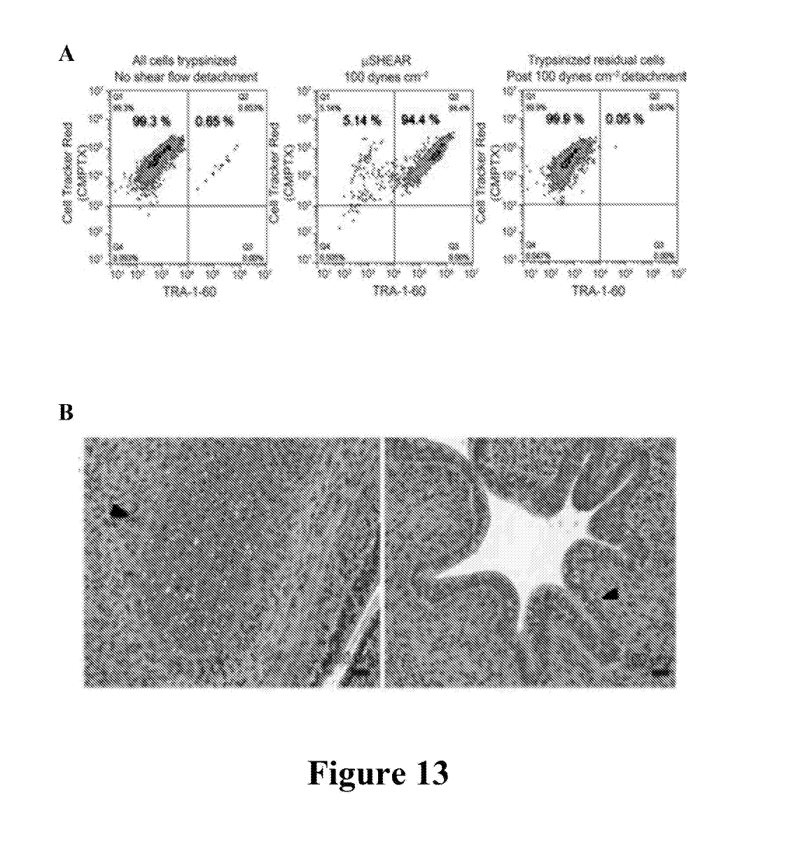

We have exploited adhesive signature differences to selectively isolate hiPSC from partially reprogrammed cultures. Using μSHEAR, we isolated hiPSC colonies (>95% purity) without detachment of non-reprogrammed and partially reprogrammed cells. We observed only 0.05% residual hiPSC, whereas non-hiPSC constituted 99.9% of the culture remaining in the μSHEAR device. Isolated hiPSC expressed TRA-1-60, TRA-1-81, DNMT3B, REX1, OCT4, SSEA4, GDF3, hTERT and NANOG (FIG. 13, Panel A), indicating that they were fully reprogrammed. μSHEAR-isolated hiPSC displayed unmethylated OCT4, SOX2 and NANOG, similarly to hiPSC under standard culture conditions. Finally, μSHEAR-isolated hiPSC formed teratomas in immunodeficient mice (FIG. 13, Panel B). These studies demonstrate that fully reprogrammed, bona fide hiPSC can be selectively isolated from parental fibroblasts and partially reprogrammed cells using μSHEAR.

### Analysis of the Adhesion Strength of Adult and Cancer Stem Cells as Well as Their Progeny and Establishment of the Ability of μSHEAR to Purify These Different Stem Cells Populations.

We will characterize the ‘adhesive force signature’ of these two diverse stem cell types to establish the broad potential of μSHEAR to purify stem cells. We will analyze the adhesive forces, integrin expression profiles, and focal adhesion (FA) assembly in various subpopulations to characterize their adhesive signatures. Human MSC, and their differentiated progeny consisting of osteoblasts, adipocytes, and chondrocytes, will be used as a representative example of adult stem cells. We will then translate this information to the μSHEAR microfluidic technology to purify undifferentiated MSC from bone marrow aspirates as well as purifying stem cells and progeny from differentiating MSC cultures. For CSC, we will use the human breast cancer cell lines MCF-7 and MDA-MB231, which contain a small population of tumorigenic cancer stem cell-like cells. After establishing the ability of the μSHEAR technology to selectively purify CSC from these breast cancer lines, we will apply the μSHEAR technology to purify CSC from fresh breast cancer human tissue.

Human stem cells, such as adult, embryonic and induced pluripotent stem cells, represent disruptive technologies for the generation of (i) auto- and allo-genic cell sources for countless therapeutic applications and (ii) novel models for the study of human development and disease. Over the last decade, huge progress has been made in establishing stem cell markers and genomic/proteomic/metabolomic profiles, developing culture conditions that maintain self-renewal capacity and direct differentiation, and discovering pathways regulating self-renewal and fate decisions. Despite these advances, efficient purification of stem cells and their progeny remains a major roadblock to widespread basic biology studies and therapeutic applications.

Current methods for ES and iPS culture rely on manual isolation, either alone or in combination with enzymatic dissociation. Such methods are time intensive, require skilled labor, are dependent on morphologic recognition of undifferentiated cells, and have been associated with karyotypic abnormalities. Label-based methodologies, such as antibody-based flow cytometry and magnetic bead sorting, are widely used for purification of virtually all stem cell types. Although these methodologies are efficient, they require the use of pre-identified probes (e.g., antibodies) specific for surface markers and dedicated instrumentation and core facilities. In many cases, robust and selective probes are not available for discriminating among parental, stem cells, and progeny. Consequently, there is a significant and unmet need for unbiased, efficient, label-free technologies for the purification of various stem cell populations such as parental/support cells, undifferentiated stem cells, partially committed/differentiated precursors, and differentiated progeny. This crucial need for robust purification technologies is relevant to adult (e.g., mesenchymal stem cells (MSC) and endothelial progenitor cells), embryonic (ES), and induced pluripotent stem (iPS) cells. In an analogous fashion, cancer stem cells (CSC) isolated from tumors exhibit self-renewal and give rise to all cell types found in a particular cancer sample. CSCs are proposed to persist in tumors as a distinct population and cause relapse and metastasis by giving rise to new tumors. The CSC field also necessitates efficient platforms to purify these stem cells and their progeny at various stages of differentiation.

We next applied the μSHEAR technology to effectively separate hiPSC from differentiated progeny. Spontaneously differentiating hiPSC cultures with varying levels of differentiation were dissociated and cultured overnight in μSHEAR devices with hiPSC. We could isolate hiPSC as intact epithelial colonies with >97% purity and yield before detaching differentiated fibroblast-like cells, and we observed similar results with hESC. We did not achieve selective purification with commonly used enzymatic agents. TRA-1-60 antibody-based purification of hiPSC yielded equivalent purification levels as μSHEAR but resulted in poor survival (<40%) as compared to μSHEAR (>80%). Furthermore, we applied μSHEAR to isolate terminally differentiated cells. Because their adhesion strength is lower than that of hiPSC, neurons were efficiently recovered whereas hiPSC remained adherent to the substrate. Isolated neurons exhibited excellent viability, neurite growth and expression of MAP2 and β-III tubulin. Similarly, we successfully isolated hiPSC-derived cardiomyocytes from hiPSC with >95% purity. Isolated cardiomyocytes expressed a-smooth muscle actin and exhibited spontaneous contractile activity.

In summary, we have shown substantial differences in adhesive force signature among human ES and iPS cells, partially reprogrammed cells, parental somatic cells and differentiated progeny. We exploited these differential adhesion strengths to rapidly (<10 min) and efficiently isolate fully reprogrammed iPSC as intact colonies from heterogeneous reprogramming cultures and from differentiated progeny using microfluidics. hiPSC were isolated label free, enriched to 95-99% purity with >80% survival, and had normal transcriptional profiles, differentiation potential and karyotypes. We also applied this strategy to isolate ES and iPSC during routine culture and showed that it may be extended to isolate iPSC-derived stem cells or differentiated cells.

### Analysis of the Adhesion Strength of Adult and Cancer Stem Cells as Well as Their Progeny and Establishment of the Ability of μSHEAR to Purify These Different Stem Cells Populations.

We will characterize the ‘adhesive force signature’ of these two diverse stem cell types and establish the broad potential of μSHEAR to purify cell subpopulations related to these stem cell types. Our general strategy has two parts: (i) characterizing the adhesive signature of a target cell population using quantitative bioengineering platforms, and (ii) translating the adhesion strength values from the adhesive signature into flow conditions in the μSHEAR microfluidics platform to purify target cells from mixed/heterogeneous populations.

Adhesive signature. To characterize the adhesive signature for each cell type/subpopulation of interest, we will analyze their adhesive force, integrin expression profile, and FA assembly on fibronectin and laminin. The adhesion strength will be measured using a custom-built spinning disk device which applies a linear range of hydrodynamic detachment forces in a single experiment and provides direct measurements of the cell-ECM adhesion strength. Because a wide range of detachment forces is applied in a single experiment, this assay provides a powerful and efficient approach to determine target adhesive forces to use in the μSHEAR microfluidics platform. Integrin expression profiles, cell spreading, and FA assembly will be analyzed using standard flow cytometry and immunostaining/image analysis methods.

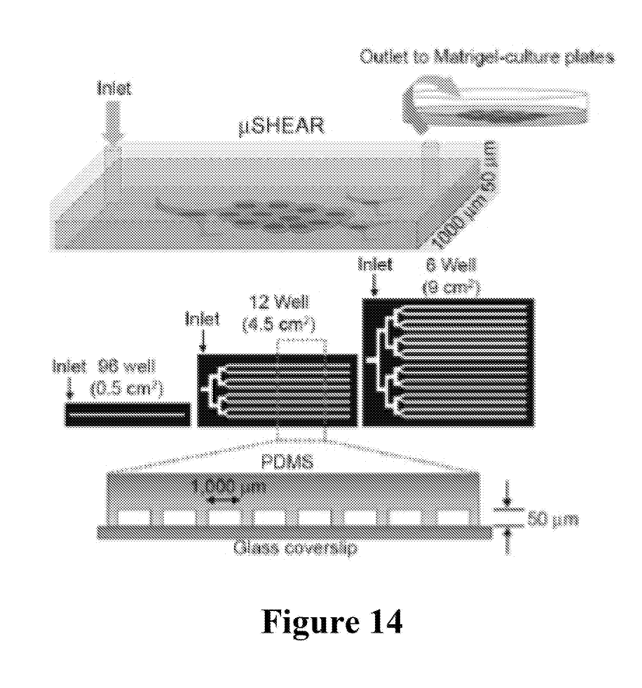

μSHEAR isolation. The adhesion strength values for each target cell values are easily translated to flow rates for use in the μSHEAR microfluidics device. Because the dimensions of the flow channel within the μSHEAR device are defined, the applied hydrodynamic force is linearly proportional to the flow rate, and by prescribing a flow rate, controlled fluid forces can be applied. The μSHEAR microfluidics device consists of a micromolded elastomeric chamber which is simply and inexpensively fabricated using PDMS biocompatible polymer and bonded to a glass slide (FIG. 14). This device can be autoclaved and easily scaled up. Importantly, flow through the device can be achieved using conventional, inexpensive syringe pumps. For μSHEAR isolation, mixed/heterogeneous cultures will be introduced into the μSHEAR device pre-coated with fibronectin or laminin and allowed to adhere. After a prescribed adhesion time (typically overnight but can be done for a few hours to weeks), buffer will be flowed through the device and detached cells will be collected at the outlet of the device and either plated on standard culture dishes or processed for analysis. Notably, the microfluidics platform allows for visualization of cell detachment process, miniaturization of sample volumes for high recovery yields and cost savings, and ability for scale-up by using parallel flow arrays.

CSC isolation. CSCs are highly significant to basic studies of disease progression and metastasis and represent promising targets for drug screening and development of new therapeutics. CSCs are presently isolated using antibody-based separations developed for other stem cells types. Therefore, the CSC field also necessitates efficient platforms to purify these stem cells and their progeny at various stages of differentiation. We will first use the human breast cancer cell lines MCF-7 and MDA-MB231, which contain a small population (<1%) of tumorigenic cancer stem cell-like cells. We will establish the adhesive signature for the CSC subpopulation in these cancer lines as well as the parental cell line. CSCs will be purified using Aldefluor-based flow cytometry and characterized by expression of Oct3/4 and CD44hi/CD241o. Based on the adhesive signature results, we will then examine the ability of μSHEAR to purify CSC from the MCF-7 and MDA-MB231 parental lines. We will compare the purification efficiency, yield, viability, proliferation, anchorage-dependent sphere-formation capacity, and tumor formation capacity (when implanted in nude mice) of μSHEAR-isolated CSC to cells isolated by commercial antibody-based sorting procedures such as R&D Systems MagCellect CD24-CD44+ Breast Cancer Stem Cell Isolation Kit.

After establishing the ability of the μSHEAR technology to selectively purify CSC from these breast cancer lines, we will apply the μSHEAR technology to isolate CSCs from fresh breast cancer human tissue. CSC will be isolated by μSHEAR, characterized as discussed above and compared to commercial antibody-based sorting procedures. We will also correlate the adhesive signature for individual samples to the pathology reports to identify any possible relationships (e.g., adhesive signature as a marker of metastatic potential or resistance to treatment) to analyze in future studies.

We will expand the μSHEAR technology to selectively and efficiently purify adult and cancer stem cells and progeny. As appropriate to address the potential limitation that the adhesion strength values for target cells are not different enough to provide reliable isolation resolution, we will modify the adhesive surface inside the microfluidic device to include micro-grooved substrates, to provide better discrimination of adhesive forces.

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is presented.

TABLE 1
Characteristics of TICs

| Property | Description |
|---|---|
| Tumor initiation | TICs have the capacity to form tumors that |
| | resemble the tumor of origin in immunodeficient |
| | hosts |
| Drug/stress | An increased resistance to stresses including |
| resistance | hypoxia, radiation, chemotherapy, treatment with |
| | other cancer drugs has been observed in TICs. This |
| | has been party attributed to an enhanced DNA |
| | damage response as well as more effective |
| | clearance of cytotoxic agents from the cell |
| Surface marker | Surface markers expression levels are widely used |
| expression | as tools for TIC purification. The markers vary |
| | widely among cancer types. |
| High ALDH | ALDH activity is increased in TICs which results in |
| activity | protection from ROS damage and increased survival |
| Sphere foiniation | TICs have an increased ability to grow and form |
| | spheroids in suspension culture |
| Pluripotent gene | The expression of pluripotent genes such as Oct4 |
| activation | and Nanog is increased. |
| Unique metabolic | Higher mitochondrial membrane potential, lower |
| activity | quantity of mtDNA, and lower intracellular |
| | concentration of ATP and ROS have been observed |
| | in TICs |
| Changed cell | The expression of adhesion proteins such as |
| adhesion | integrins is dysregulated, resulting in a changed cell |
| | adhesion profile |
| Decreased cell | Decreased cell stiffness and increased deformability |
| stiffness | have been observed in TICs |
| Differential | The increased activity of the ABC transporter |
| Hoechst | results in differential staining of TICs by Hoechst |
| 33342 staining | 33342, allowing for isolation by SP staining |

TABLE 2
Common TIC surface markers

| Tumor type | Markers |
|---|---|
| Breast | CD44⁺/CD24^{low}, CD133⁺, CD44⁺/CD176⁺, ESA⁺ |
| | (EpCam⁺), CD24⁺/CD29⁺, CD24⁺/CD49f⁺ |
| Colorectal | EpCAM^{high}/CD44⁺, CD133⁺ |
| Liver | CD90⁺, CD44⁺/CD176⁺, CD133⁺, CD13⁺ |
| Pancreatic | CD44⁺CD24⁺ESA⁺, CD133⁺, CXCR4⁺ |
| Ovarian | CD133+, CD44⁺CD117⁺, CD24⁺ |
| Prostate | CD44⁺/α₂β₁^{hi}/CD133⁺, |
| Bladder | CD44⁺CK5⁺CK20⁻ |
| Lung | CD176⁺, CD133⁺, CD44⁺ |
| Brain | CD133⁺, SSEA-1⁺ |
| Melanoma | CD20⁺, CD166⁺, CD133⁺, ABCB5⁺ |
| Gastric | CD44⁺, CD133⁺ |
| Osteosarcoma | CD133⁻, CD117⁺, Stro-1⁺ |

1. A method of isolating a cancer-associated cell from a mixture of cultured animal cells, comprising subjecting a mixture of cultured animal cells adhered to a substrate comprising the cancer-associated cell and at least one other cell type to a detachment force that is sufficient to selectively detach the cancer-associated cell from the substrate relative to the at least one other cell type in the mixture of cultured animal cells, thereby isolating the cancer-associated cell from the mixture of cultured animal cells.

2. The method of claim 1, wherein the cancer-associated cell is a cancer stem cell.

3. The method of claim 1, wherein the cancer-associated cell is a tumor initiating cell.

4. The method of claim 1, wherein the cancer-associated cell is a cancer stem-like cell.

5. The method of claim 1, wherein the at least one other cell type is a stem cell, a progenitor cell, a terminally differentiated cell, a stromal cell, an inflammatory cell, an explant cell and/or a progeny cell of any of said cells.

6. The method of claim 1, wherein the cancer-associated cell grows in culture as part of a cluster.

7. The method of claim 6, wherein the cancer-associated cell detaches from the substrate as part of a cluster of cancer-associated cells.

8. The method of claim 1, wherein the detachment force that is sufficient to selectively detach the cancer-associated cell provides a wall shear stress in the range of 20 to 1500 dynes/cm².

9. The method of claim 1, wherein the cancer-associated cell detaches at a lower detachment force as compared with the at least one other cell type.

10. The method of claim 1, wherein the cancer-associated cell detaches at a higher detachment force as compared with the at least one other cell type.

11. The method of claim 1, wherein the isolated cancer-associated cell is viable.

12. The method of claim 11, wherein the isolated cancer-associated cell maintains the ability to divide and produce progeny cells and/or form tumors.

13. The method of claim 1, wherein a plurality of cancer-associated cells is isolated with at least 80% purity.

14. The method of claim 1, wherein at least 70% of⁻the cancer-associated cells in the mixture of cultured animal cells are isolated.

15. The method of claim 1, wherein the cultured animal cells are mammalian cells, optionally human cells.

16. The method of claim 1, wherein the method further comprises culturing the isolated cancer-associated cell.

17. The method of claim 1, wherein the method further comprises evaluating the isolated cancer-associated cell by flow cytometry, biochemical analysis, mammosphere assay, tumorsphere assay, migration assay, tumor formation assay, and/or gene expression analysis.

18. The method of claim 1, wherein the method does not comprise attaching a detectable label and/or affinity reagent to the mixture of cultured animal cells.

19. The method of claim 1, wherein the detachment force is applied by hydrodynamic force, centrifugal force and/or magnetic force.

20. The method of claim 1, wherein the method is carried out in a fluid flow device.

21. The method of claim 1, wherein the mixture of cultured animal cells is subjected to the detachment force for 1 to 60 minutes.

22. The method of claim 1, wherein the mixture of cultured animal cells is subjected to the detachment force for 2 to 20 minutes.

23. The method of claim 1, wherein the cancer-associated cell is from a cancer selected from the group consisting of melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, stomach cancer, esophageal cancer, and brain cancer.

24. The method of claim 1, wherein the source of the cultured animal cells is a cancer cell line.

25. The method of claim 1, wherein the source of the cultured animal cells is primary tumor tissue.
